(19) United States
(12) Patent Application Publication
(10) Pub. No.: US 2006/0135587 A1
(43) Pub. Date: Jun. 22, 2006

(54) Method and compounds for promoting healing and reducing inflammation

(76) Inventors: Nikolay Sergeevich Sapronov, St. Petersburg (RU); Levon Borisovich Piotrovsky, St. Petersburg (RU); Luidmila Konstantinovan Gavrovskaya, St. Petersburg (RU)

Correspondence Address: HAYNES AND BOONE, LLP, 901 MAIN STREET, SUITE 3100, DALLAS, TX, 75202, US

(21) Appl. No.: 10/519,645

(22) PCT Filed: Jul. 31, 2003
(86) PCT No.: PCT/AU03/00972

§ 371 Date: Sep. 22, 2005

(30) Foreign Application Priority Data
Aug. 1, 2002 (RU) 2002120366

(51) Int. Cl. A61K 31/4172

(52) U.S. Cl. 514/400

(57) ABSTRACT

This invention relates to methods of promoting healing and reducing inflammation, and compositions therefore. In particular, the invention relates to the use of 1,3-dialkyl-4,5-bis(N-methylcarbamoyl)imidazolium salts to promote wound healing and to reduce inflammation. Novel compounds and compositions are also provided. In one preferred embodiment the invention provides a method of treatment of myocardial infarction.

This invention relates to methods of promoting healing and reducing inflammation, and compositions therefore. In particular, the invention relates to the use of 1,3-dialkyl-4,5-bis(optionally N-substituted carbamoyl)imidazolium salts to promote healing and to reduce inflammation. In one preferred embodiment the compounds are useful in the treatment of myocardial infarction. Novel compounds and compositions are also provided.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Tissue or organ damage in mammals is usually accompanied by inflammation. Inflammation is a complex process associated with the activity of inflammatory mediators such as histamine, serotonin, bradykinin, prostaglandin and other biologically active substances, and may be caused by a variety of endogenous or exogenous pathological agents or processes. The inflammatory process may result in the malfunction of specific organs and in deterioration of the overall state of health.

Both non-steroidal anti-inflammatory agents (NSAIDS) and steroid hormones such as corticosteroids have an anti-inflammatory effect, and are widely used in medical practice. Corticosteroids have serious side-effects, including excessive accumulation of sodium and water in the body, oedema, increased blood pressure, decreased resistance to infection, ulceration of the gastrointestinal mucosa, impaired wound healing and tissue regeneration, susceptibility to blood clotting, and obesity, as well as endocrine, nervous and psychiatric disorders. If corticosteroids are administered over a long period, there is a risk that the production of natural hormones by the adrenal glands can be suppressed.

The anti-inflammatory activity of NSAIDS is due to their suppressive effect on the enzymes involved in the synthesis of prostaglandin (prostaglandin synthetase), serotonin (5-hydroxytryptophan decarboxylase), histamine (histamine decarboxylase) and on the release of inflammatory mediators. NSAIDS may cause side-effects such as irritation of the gastric mucosa, allergic reactions, and liver and kidney dysfunction.

Wound healing is a complex morphological, pathophysiological and biochemical process whose progress and outcome are significantly affected by a variety of factors relating to skin injury. The general wound healing process involves three stages:

1. Degradation of necrotic pulp and removal of necrotic material from the wound defect via an inflammatory process;

2. Proliferation of connective tissue elements and formation of granulation tissue to fill the wound defect; and

3. Fibrosis of the granulation tissue, and formation and epithelialization of scar tissue.

Many agents have been proposed for stimulation of wound healing and skin regeneration, including hormones, enzymes, plant-derived agents, and polypeptide growth factors.

Wounds can result not only from trauma, including burns, but also from surgical interventions. In particular, plastic reconstructive surgery and dermatological techniques can result in skin defects which require rapid healing with no or minimal scar formation. These techniques include procedures such as laser skin resurfacing, blepharoplasty, dermabrasion, chemical peeling and the like. During the post-operative period, patients may suffer complications such as prolonged erythema, dermatitis, hyperpigmentation, or infection with organisms such as Pseudomonas aeruginosa, Staphyloccoccus aureus, S. epidermidis, some Gram-negative bacteria, various types of Candida, or the virus Herpes simplex. This may result in formation of hypertrophic scars, and may necessitate a long treatment period (Sriprachya-Anunt et al, 1997).

While these complications may be reduced or avoided by careful post-operative care and the use of anti-infective agents and promoters or tissue repair and regeneration, complete avoidance of complications is not always possible.

To heal skin wounds, various drugs may be used as stimulators of the tissue regeneration process, including proteolytic enzymes, synthetic anabolic hormones, growth factors, antiseptics, honey and its products, phytomedicinals, various oils, adsorbents, etc (Mashkovsky, 1998).

In the post-operative period antibiotics, antiviral drugs, corticosteroids, and vitamins such as vitamins A, C, and E may be used in conjunction with drugs enhancing tissue repair and regeneration (Katzung, 1998).

Thus there is still a considerable need in the art for methods and agents for effective and low cost stimulation of wound healing and reduction of scar formation.

The compound ethimizole (1-ethylimidazole-4,5-dicarboxylic acid bis-N-methylamide; 1-ethyl-4,5-di(N-methylcarbamoyl)imidazole) 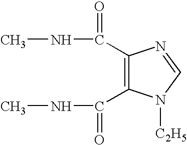
has been used as an antiallergic and anti-inflammatory medicinal agent in the treatment of arthritis, inflammatory polyarthritis, and certain forms of bronchial asthma (M. D. Mashkovsky, Medicinals, Medicine, 1987, pp. 130-131). Under biological conditions, the uncharged imidazole ring of imidazole-4,5-dicarboxylic acid enables ethimizole to penetrate through the blood-brain barrier.

Consequently this drug causes undesirable side effects associated with its influence on the central nervous system. In particular, ethimizole is contraindicated for patients with motor or psychological agitation. Ethimizole has not been suggested to be useful for treatment of wounds.

In our previous patent RU1075668, we disclosed 1,3-dialkyl-4,5-bis(N-methylcarbamoyl)imidazole benzene sulphonate compounds which had a stimulating effect on tissue energy metabolism. These compounds had the general formula 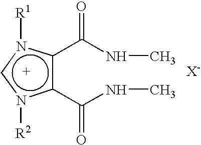
in which R¹ and R² may be the same or different, and are independently selected from methyl and ethyl, and X⁻ is benzenesulphonate. In these compounds the imidazole ring of ethimizole is replaced by a charged imidazolium ring. Such charged compounds are unable to penetrate through the blood-brain barrier, and therefore cannot affect the central nervous system.

The specification of RU1075668 disclosed the synthesis of three compounds within the formula, namely the 1,3-dimethyl,1-methyl-3-ethyl, and 1,3-diethyl compounds, and their ability to prevent development of neurogenic gastric lesions in rats, to promote healing of such lesions, and to increase creatine phosphate levels in the gastric wall, when administered intra-peritoneally. It was suggested that, because of this stimulatory effect on energy metabolism, the compounds might be useful as tissue repair agents.

However, no guidance at all was provided as to how any other condition could be treated, how the compounds should be formulated, or by what routes they should be administered. Only intra-peritoneally administration was disclosed. In particular, there was no general disclosure or suggestion that any of the compounds disclosed in this specification could have any activity in promoting healing of wounds, burns, skin ulcers or the like, in reducing scar formation, in reducing inflammation, in stimulating repair of bone, or in treating myocardial infarction.

We have now found that 1,3-dialkyl-4,5-bis(N-methylcarbamoyl)imidazolium salts promote tissue repair in a variety of settings, and in particular promote wound healing and reduce scar formation. We have also found that that a number of 1,3-dialkyl-4,5-bis(N-methylcarbamoyl)imidazolium salts possess anti-inflammatory and wound healing properties, and that the compounds are active both orally and topically. These compounds demonstrate an anti-inflammatory effect in experimental models of inflammation, have no toxic effects in a variety of assays, and are readily synthesised using simple reaction schemes.

Without wishing to be limited by any proposed mechanism, we believe that their anti-inflammatory activity is due to their suppressive effect on the synthesis and secretion of mediators of inflammation.

### SUMMARY OF THE INVENTION

In a first general aspect, the invention provides a method of promoting tissue repair or wound healing, comprising the step of administering an effective amount of a 1,3-dialkyl-4,5-bis(optionally N-substituted carbamoyl)imidazolium salt to a subject in need of such treatment.

In one preferred embodiment, the invention provides a method of reducing inflammation, comprising the step of administering an effective amount of a 1,3-dialkyl-4,5-bis (optionally N-substituted carbamoyl)imidazolium salt to a subject in need of such treatment.

In a second preferred embodiment, the invention provides a method of reducing scar formation, comprising the step of administering an effective amount of a 1,3-dialkyl-4,5-bis (optionally N-substituted carbamoyl)imidazolium salt to a subject in need of such treatment.

In a third preferred embodiment, the invention provides a method of treatment of myocardial infarction, comprising the step of administering an effective amount of a 1,3-dialkyl-4,5-bis (optionally N-substituted carbamoyl)imidazolium salt to a subject in need of such treatment.

In a fourth preferred embodiment, the invention provides a method of stimulating bone repair, comprising the step of administering an effective amount of a 1,3-dialkyl-4,5-bis (optionally N-substituted carbamoyl)imidazolium salt to a subject in need of such treatment.

In a fifth preferred embodiment, the invention provides a method of treatment of ulcerative colitis, comprising the step of administering an effective amount of a 1,3-dialkyl-4,5-bis (optionally N-substituted carbamoyl)imidazolium salt to a subject in need of such treatment.

Preferably the 1,3-dialkyl-4,5-bis (optionally N-substituted carbamoyl)imidazolium salt is a compound of formula I

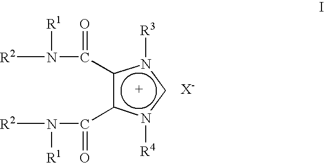

in which R¹ and R² may be the same or different, and each is selected from the group consisting of hydrogen and a linear or branched alkyl group of 1 to 6 carbon atoms, which may optionally be substituted by an amino, substituted or unsubstituted aminomethyl, nitro, hydroxyl, halogen, carboxy, or carboxylic acid amide group;

R³ and R⁴ may be the same or different, and each is a linear or branched alkyl group of 1 to 6 carbon atoms; and

X⁻ is a pharmaceutically acceptable inorganic or organic anion selected from the group consisting of chloride, bromide, iodide, sulphate, nitrate, phosphate, perchlorate, formate, acetate, fumarate, malate, malonate, citrate, benzoate, salicylate, benzenesulfonate, methylsulfonate, p-toluenesulfonate, gentisate (dihydroxybenzoate), and naphthalene-8-sulfonate.

When R¹ or R² is a halogen, this halogen is chlorine, bromine or iodine.

Preferably R¹ and R² are different, for example if R¹ is hydrogen, R² is R² is an alkyl group with 1 to 6 carbon atoms; R³ and R⁴ are the same or different, and are each independently an alkyl group with 1 to 6 carbon atoms, more preferably 1 to 4 carbon atoms. Even more preferably R³ is methyl and R⁴ is ethyl.

The anion X⁻ has no specific limitations, and is preferably an organic anion selected from the group consisting of benzenesulfonate, methylsulfonate, n-toluenesulfonate, formate, acetate, fumarate, malate, malonate, citrate, benzoate, salicylate, gentisate, and naphthalene-8-sulfonate. More preferably X⁻ is benzenesulfonate, benzoate, salicylate, or gentisate. Most preferably X⁻ is benzenesulphonate. Alternatively X⁻ is preferably an inorganic anion selected from the group consisting of chloride, bromide, and iodide.

R³ and/or R⁴ may optionally be substituted with a group selected from a substituted or unsubstituted amino, substituted or unsubstituted aminomethyl, NO₂, acetamide, or substituted or unsubstituted sulphonamide group. Preferably at least one of R³ and R⁴ is unsubstituted; more preferably both R³ and R⁴ are unsubstituted. Where R³ or R⁴ is a substituted sulphonamide, the substituent is preferably an alkyl chain of 1 to 6, more preferably 1 to 4 carbon atoms.

Particularly preferred compounds for use in the method of the invention are:
1,3-dimethyl-4,5-bis(N-methylcarbamoyl)imidazolium benzenesulfonate (1),
1-methyl-3-ethyl-4,5-bis(N-methylcarbamoyl)imidazolium benzenesulfonate (2),
1,3-diethyl-4,5-bis(N-methylcarbamoyl)imidazolium benzenesulfonate (3),
1-methyl-3-ethyl-4,5-bis(N-methylcarbamoyl)imidazolium chloride (4)
1-methyl-3-ethyl-4,5-bis(N-methylcarbamoyl)imidazolium benzoate (5),
1-methyl-3-ethyl-4,5-bis(N-methylcarbamoyl)imidazolium salicylate (6), and
1-methyl-3-ethyl-4,5-bis(N-methylcarbamoyl)imidazolium gentisate (7).

The method of the invention is applicable to the treatment of damage to epithelial, mucosal, muscular, cardiac, liver, and bone tissue, caused by erosions, ulcers, chronic injury, infection, trauma or surgery. For example, the damage may be gastric or duodenal ulcers, myocardial infarction, liver conditions such as cirrhosis and hepatitis, or bone fractures.

Wounds arising from a wide variety of causes can be treated using the method of the invention, including but not limited to traumatic wounds, surgical wounds, burns, dehisced surgical incisions, grafts, diabetic ulcers, varicose ulcers, decubitus ulcers (bedsores), trophic ulcers, tropical ulcers, steroid ulcers, indolent ulcers, oral or pharyngeal ulcers, aphthous ulcers, and corneal ulcers; gastric, duodenal, and peptic ulcers; ulcerative colitis; cervical erosions; myocardial damage, including myocardial infarction; liver damage, for example caused by cirrhosis or hepatitis; and bone fractures.

In all embodiments of the first aspect of the invention, the 1,3-dialkyl-4,5-bis(optionally N-substituted carbamoyl)imidazolium salt is preferably administered orally or, if appropriate for the specific condition, topically or via enema.

In a second aspect, the invention provides a compound of formula I as defined above, but with the proviso that when X⁻ is benzenesulphonate, R¹ is hydrogen and R² is methyl, then R³ and R⁴ are not methyl or ethyl.

In a third aspect, the invention provides a composition comprising a compound according to the invention, together with a pharmaceutically or veterinarily acceptable carrier. We have unexpectedly found that the compounds of the invention are active when administered orally or topically. Therefore in one preferred embodiment the composition is adapted for oral administration. In a second preferred embodiment the composition is one which is adapted to topical administration, such as an ointment, cream or gel.

Methods and pharmaceutical carriers for preparation of pharmaceutical compositions are well known in the art, as set out in textbooks such as Remington's Pharmaceutical Sciences, 19th Edition, Mack Publishing Company, Easton, Pa., USA.

Examples of formulations which can be used for the purposes of the invention include topical formulations such as creams, gels, ointments, and impregnated bandages; rectal or vaginal formulations such as tampons, suppositories and pessaries; oral formulations such as tablets, lozenges, capsules or solutions; buccal or sublingual formulations such as tablets or lozenges; solution or spray formulations for intranasal use; enemas for rectal use; solution formulations for injection; and cellulose or collagen dressings.

It will be clearly understood that the proviso to formula I set out in the second aspect of the invention applies only to simple solution formulations in which the carrier is water or saline. It does not apply to the first aspect of the invention, or to compositions adapted for topical or oral administration.

The mammal to be treated may be a human, or may be a domestic or companion animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

It will be clearly understood that the method of the invention may be used in conjunction with one or more other treatments, such as other therapeutic agents or the use of hyperbaric oxygen or subatmospheric pressure.

In a fourth aspect the invention provides a method of synthesis of a compound of formula I, comprising the step of subjecting an 1-alkyl-4,5-bis(optionally N-substituted carbamoyl)imidazole to alkylation (quaternization) with an alkyl benzenesulfonate to produce the corresponding imidazolium benzenesulfonate, and optionally replacing the benzenesulfonate anion by ion exchange, in which the imidazole moiety is as defined in formula I.

For the purposes of this specification it will be clearly understood that the word “comprising” means “including but not limited to”, and that the word “comprises” has a corresponding meaning.

### BRIEF DESCRIPTION OF THE FIGURES

FIG. 1 illustrates the healing of skin full-thickness wounds at 15 days.
A: control group, self-healing without treatment;
B: experimental group treated with compound (2) (10% cream);
C: control group treated with Spasatel balm;
D: experimental group treated with Solcoseryl gel.

FIG. 2 illustrates the healing of cryogenic damage to the mucous membrane of the mouth.
A: controls, cryogenic damage—7 days;
B: experimental group treated with compound (2), 50 mg/kg—7 days;
C: experimental group treated with compound (2), 50 mg/kg—14 days.

FIG. 3 compares the effect of pharmacological agents on gastric ulcer in rats following electrostimulation. The vertical axis represents the number of ulcers per animal:
1. Control
2. Animals treated with 50 mg/kg methyl uracil.
3. Animals treated with 12 mg/kg cimetidine.
4. Animals treated with mg/kg Compound 2.
Panel A: Intraperitoneal administration; Panel B: oral administration.

FIG. 4 illustrates the healing of cryogenic ulcers of the large intestine, at 7 days.
A: controls, cryogenic ulcer;
B: experimental group treated with compound (2), 20 mg/kg;
C: experimental group treated with compound (2), 50 mg/kg;
D: experimental group treated with methyl uracil 180 mg/kg.

FIG. 5 shows the effect of Compound 2 on cyclic AMP levels (panel A) and Ca⁺⁺ levels (panel B) in rabbit myocardial tissue.
1. Untreated control.
2. Electrostimulation alone.
3. 10 mg/kg Compound 2 prior to electrostimulation
4. Compound 2 given 3 days after electrostimulation.
5. Compound 2 daily for 3 days after electrostimulation.

FIG. 6A shows creatine kinase levels in rabbit myocardium (a) and blood plasma (b) after electrostimulation of the aortic arch.

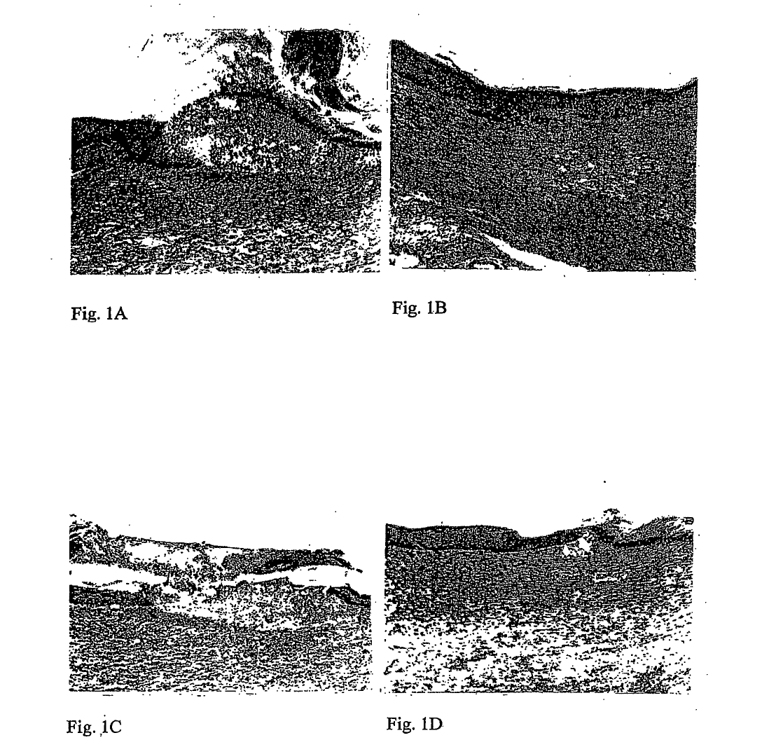

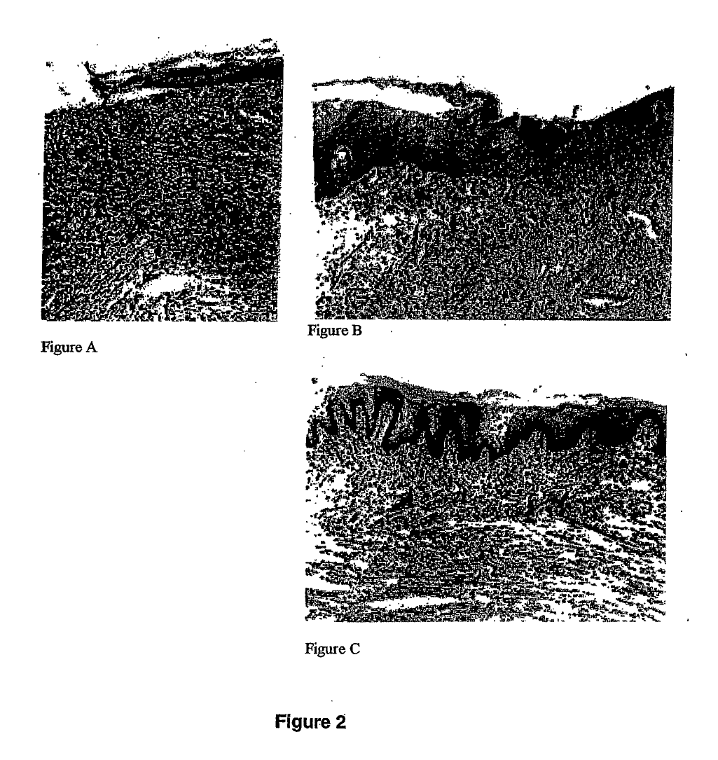

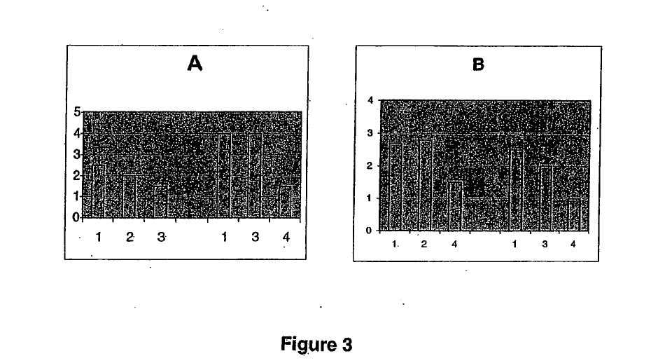

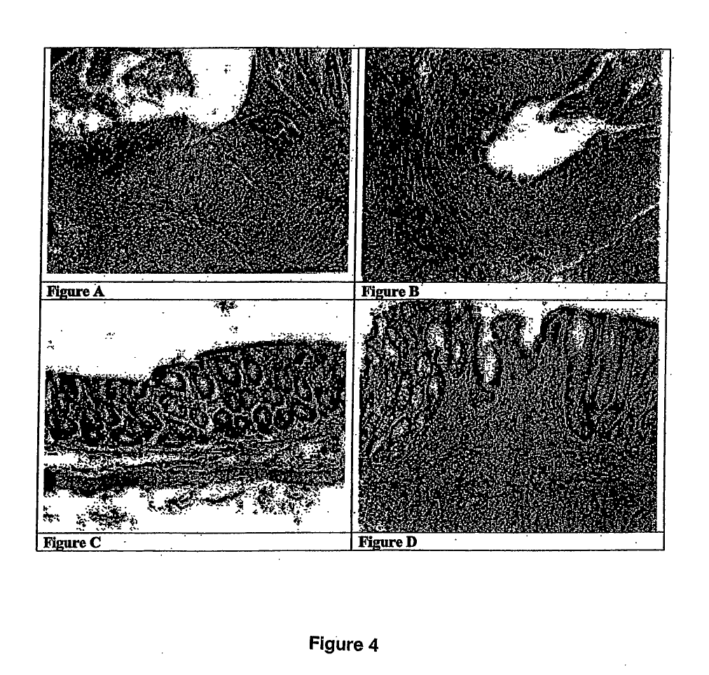

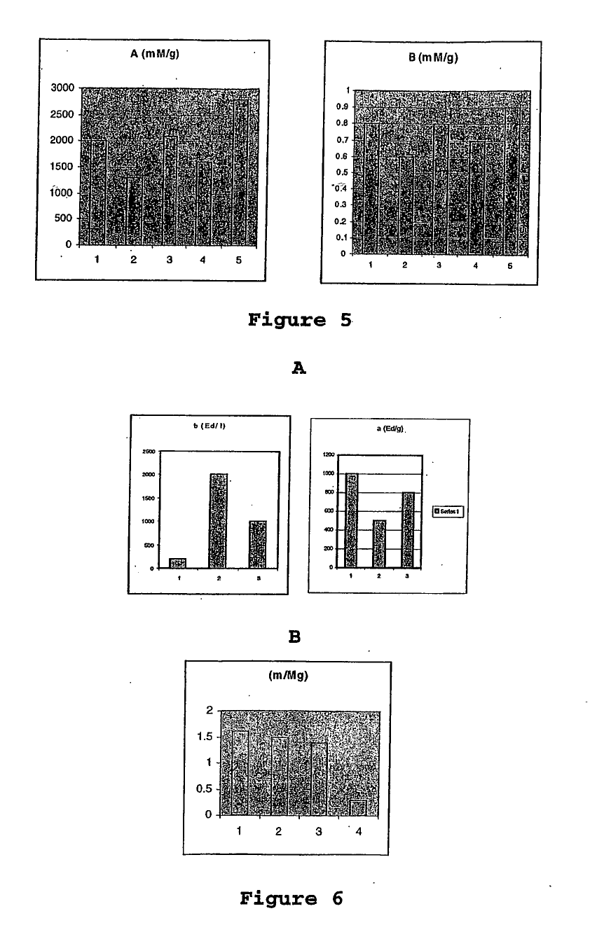

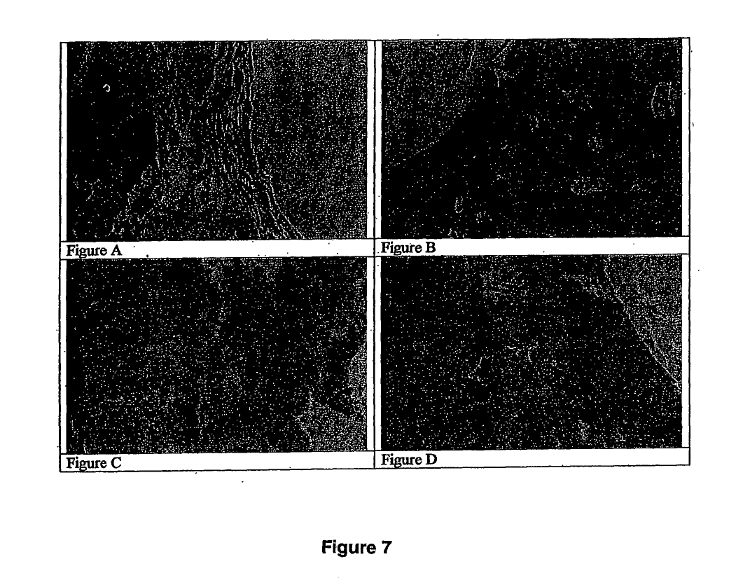

FIG. 6B shows the effect of Compound 2 on creatine phosphate levels in the myocardium. In both FIGS. 6A and 6B:
1. Untreated control animals.
2. Electrostimulation alone.
3. Animals treated with 10 mg/kg Compound 2.
4. Animals treated with 10 mg/kg riboxine.

FIG. 7 illustrates regeneration of bone tissue in the area of an artificially-created defect in the bone plate of the lower jaw of rats.
A: controls, self-healing without treatment—1 month;
B: experimental group treated with compound (2) 50 mg/kg—3 months;
C: controls, self-healing without treatment—3 months;
D: experimental group treated with compound (2) 100 mg/kg—3 months.

### DETAILED DESCRIPTION OF THE INVENTION

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids. Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are now described.

The term “alkyl” denotes straight chain, branched or cyclic alkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, and the like.

Examples of cyclic alkyl include mono- or polycyclic alkyl groups such as cyclopropyl, cyclobutyl, cyclopentyl, cyclohexyl, and the like.

The expression “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, carboxy, benzyloxy, haloalkoxy, haloalkenyloxy, haloaryloxy, nitro, nitroalkyl, nitroalkenyl, nitroalkynyl, nitroaryl, nitroheterocyclyl, azido, amino, alkylamino, alkenylamino, alkynylamino, arylamino, benzylamino, acylamino, acyl, alkenylacyl, alkynylacyl, arylacyl, acylamino, acyloxy, aldehydo, alkylsulphonyl, arylsulphonyl, sulphonylamino, alkylsulphonylamino, arylsulphonylamino, alkylsulphonyloxy, arylsulphonyloxy, heterocyclyl, heterocycloxy, heterocyclylamino, haloheterocyclyl, alkylsulphenyl, arylsulphenyl, carboalkoxy, carboaryloxy, mercapto, sulfonic acid, alkylthio, arylthio and acylthio.

Generally, the terms “treating”, “treatment” and the like are used herein to mean affecting a subject, tissue or cell to obtain a desired pharmacological and/or physiological effect. The effect may be prophylactic in terms of completely or partially preventing a disease injury or a sign or symptom thereof, and/or may be therapeutic in terms of a partial or complete cure of a disease. “Treating” as used herein covers any treatment of, or prevention of disease or injury in a vertebrate, a mammal, particularly a human, and includes preventing the disease from occurring in a subject who may be predisposed to the disease, but has not yet been diagnosed as having it; inhibiting the disease, ie., arresting its development; or relieving or ameliorating the effects of the disease, ie., cause regression of the effects of the disease.

As used herein, the term “therapeutically effective amount” means an amount of a compound of the present invention effective to yield a desired therapeutic response, for example to prevent or treat a disease which is susceptible to treatment by administration of a pharmaceutically-active agent.

The specific “therapeutically effective amount” will, of course, vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutical carrier” is a pharmaceutically acceptable solvent, suspending agent, excipient or vehicle for delivering the compound of formula I and/or pharmaceutically-active agent to the subject. The carrier may be liquid or solid, and is selected with the planned manner of administration in mind.

The compound of formula I and/or second pharmaceutically-active agent may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

The invention also provides suitable topical, oral, aerosol, and parenteral pharmaceutical formulations for use in the novel methods of treatment of the present invention. The compounds of the invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may be inert diluents, such as calcium carbonate, lactose, calcium phosphate or sodium phosphate; granulating and disintegrating agents, such as corn starch or alginic acid; binding agents, such as starch, gelatin or acacia; or lubricating agents, such as magnesium stearate, stearic acid or talc. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in U.S. Pat. Nos. 4,256,108; 4,160,452; and 4,265,874 to form osmotic therapeutic tablets for control release.

For in vivo application, the compounds of the invention or additional pharmaceutically active agents can be administered parenterally by injection or by gradual perfusion over time, independently or together. Administration may be intravenously, intra-arterial, intraperitoneally, intramuscularly, subcutaneously, intracavity, or transdermally. For in vitro studies the compounds may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers such as those based on Ringer's dextrose, and the like. Preservatives and other additives may also be present, such as anti-microbials, anti-oxidants, chelating agents, growth factors and inert gases and the like.

The invention includes various pharmaceutical compositions useful for ameliorating disease or injury. The pharmaceutical compositions according to one embodiment of the invention are prepared by bringing a compound of formula I, or an analogue, derivative or salt thereof, and one or more pharmaceutically-active agents or combinations of a compound of formula I and one or more other pharmaceutically-active agents, into a form suitable for administration to a subject, using carriers, excipients and additives or auxiliaries.

Carriers and formulations are described, for instance, in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described in Langer, Science, 249: 1527, (1990). Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) a naturally occurring phosphatide such as lecithin;

(b) a condensation product of an alkylene oxide with a fatty acid, for example, polyoxyethylene stearate;

(c) a condensation product of ethylene oxide with a long chain aliphatic alcohol, for example, heptadecaethylenoxycetanol;

(d) a condensation product of ethylene oxide with a partial ester derived from a fatty acid and hexitol such as polyoxyethylene sorbitol monooleate, or

(e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of formula I of the present invention will usually be of the order of about 0.5 mg to about 200 mg per kilogram body weight, with a preferred dosage range between about 0.5 mg to about 100 mg per kilogram body weight per day (from about 0.5 g to about 30 g per patient per day). The amount of active ingredient which may be combined with the carrier materials to produce a single dosage will vary, depending upon the host to be treated and the particular mode of administration. For example, a formulation intended for oral administration to humans may contain about 5 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other compounds to provide an operative combination. It is intended to include any chemically compatible combination of pharmaceutically-active agents, as long as the combination does not eliminate the activity of the compound of formula I of this invention.

The methods of this invention may involve the administration of a compound of formula I, prior to, together with, or subsequent to the administration of a second pharmaceutically-active agent; or the administration of a combination of a compound of formula I and a second pharmaceutically-active agent.

In certain preferred embodiments, the invention provides new quaternary imidazolium derivatives, namely: 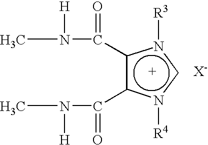
in which R¹ and R² are methyl or ethyl alkyl groups, and X⁻ is an organic or non-organic acid anion. Particularly preferred compounds are those in which:
R¹ and R² are CH₃, X⁻ is benzenesulphonate (1);
R¹ is CH₃, R² is C₂H₅, X⁻ is benzenesulphonate (2);
R¹ and R² are C₂H₅, X⁻ is benzenesulphonate (3);
R¹ is CH₃, R² is C₂H₅, X⁻ is Cl (4);
R¹ is CH₃, R² is C₂H₅, X⁻ is benzoate (5);
R¹ is CH₃, R² is C₂H₅, X⁻ is salicylate (6);
R¹ is CH₃, R² is C₂H₅, X⁻ is dihydroxybenzoate (gentisate) (7).

Compounds 1-3 can be synthesised by quaternization of 1-alkyl-4,5-bis (N-methylcarbamoyl)imidazolium on heating with an alkyl ester of benzenesulphonate, in, accordance with the following reaction: 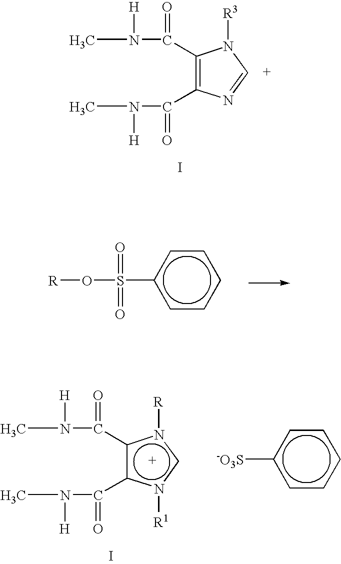
Compounds 4-7 can be synthesised by replacing the anion in 1-methyl-3-ethyl-4,5-bis(N-methylcarbamoyl)-imidazolium benzenesulfonate (2) by ion-exchange, in accordance with the following scheme:

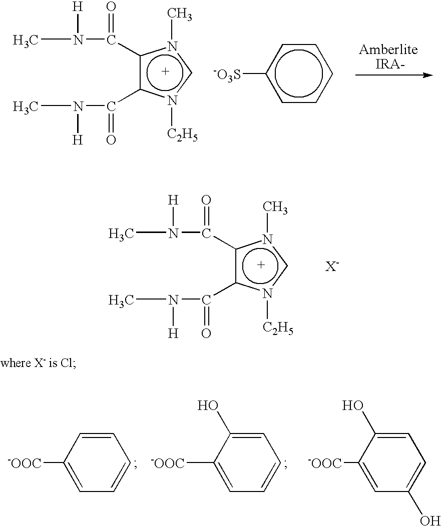

The compounds of the invention are readily soluble in water, stable, and non-toxic.

Because inflammation is a complex process consisting of several stages and involving inflammatory mediators, a uniform model of inflammation is not feasible. The effect of 1,3-dialkyl-4,5-bis (optionally N-substituted carbamoyl)imidazolium salts was assessed on models which are widely used in researching the anti-inflammatory properties of medications. The effect of the compounds of the invention was assessed with regard to both preventive and therapeutic treatments.

Experimental inflammation in mice and rats was induced by various methods. As models of acute inflammation we used induction of paw oedema in response to the administration of concanavalin A (Con A; Serva), carrageenin (Serva) or bradykinin (Sigma). As a model of chronic inflammation we used cotton wool-induced granuloma. Our results demonstrated that the compounds prevent acute inflammation caused by various inflammatory agents (prophylactic effect) and reduce the development of chronic inflammation (treatment effect), to a degree comparable to the effect of well-known anti-inflammatory medications such as non-steroidal anti-inflammatory agents.

The acuteness of the inflammation was assessed 30 minutes after the injection of bradykinin and 3 hours after injection of carrageenin respectively, by measuring the change in paw volume. The anti-inflammatory effect was assessed by the reduction of swelling was expressed in % relative to controls, or the reaction index of the ankle joint inflammation was assessed by weighing the experiment and control paws. The test substances were administered orally via an intra-gastric tube one hour before carrageenin or bradykinin was administered. As comparative medications, the following non-steroidal anti-inflammatory agents were also administered orally: butadione 60 mg/kg, ibuprofen 48 mg/kg and paramidine 50 mg/kg. These dosages correspond to ED₅₀ values from the literature.

For inflammation induced by carrageenin or bradykinin, the test substances were injected 4 times, daily for 3 days and 1 hour immediately before the administration of the inflammatory agent. For concanavalin A, the test substances were administered once, one hour prior to administration of Con A. The results obtained confirm that the compounds of the invention can reduce acute inflammation caused by concanavalin A, carrageenin or bradykinin in intact animals, both when administered in a single dose or continuously, and in adrenalectomized as well as in intact animals.

It is well known that when medications are applied to the skin, their physical and chemical characteristics, such as ionisation ability (pKa) at the pH of the structural elements of the skin and their lipophilicity (logP) affect the rate at which the active agents are released from the medication and absorbed into the skin. Because the compounds of the invention have low lipophilicity (logP approximately −0.95), they penetrate biological membranes poorly, thus helping to prolong contact with the damaged area. Both these factors prolong the effect of the compounds, increasing their efficiency when applied locally. Therefore, these agents are suitable for topical application, for example as an ointment, solution, or finely dispersed powder.

The invention will now be described in detail by way of reference only to the following non-limiting examples and drawings.

### EXAMPLE 1

### 1,3-dimethyl-4,5-bis(N-methylcarbamoyl)-imidazolium benzenesulfonate (1)

1 g of 1-methyl-4,5-bis(N-methylcarbamoyl)imidazolium [bis(N-methylamide) 1-methylimidazolium-4,5-dicarboxylic acid] was heated in 5 ml of methyl ether of benzenesulphonic acid at 120° C. for 3 hours. The reaction mixture was diluted with an appropriate solvent, such as water-free diethyl ether or water-free acetone, and the residue was filtered. The yield was 1.5g (85.7%). The melting point was 159-161° C. (n-butanol n-heptane). The results of elemental analysis and NMR spectroscopy are set out in Tables 1 and 2 respectively.

### EXAMPLE 2

### 1-methyl-3-ethyl-4,5-bis(N-methylcarbamoyl)-imidazolium benzenesulfonate (2)

1 g of 1-methyl-4,5-bis(N-methylcarbamoyl)-imidazolium [bis(N-methylamide) 1-ethylimidazolium-4,5-dicarboxylic acid] was heated in 5 ml of benzenesulphonic acid methyl ether at 120° C. for 3 hours. The reaction mixture was diluted by an appropriate solvent, such as water-free diethyl ether or water-free acetone, and the residue was filtered. The yield was 1.5 g (82.5%). The melting point was 133-135° C. (n-butanol n-heptane). The results of elemental analysis and NMR spectroscopy are set out in Tables 1 and 2 respectively.

### EXAMPLE 3

### 1,3-diethyl-4,5-bis(N-methylcarbamoyl)-imidazolium benzenesulfonate (3)

1 g of 1-ethyl-4,5-bis(N-methylcarbamoyl)-imidazolium [bis(N-methylamide) 1-ethylimidazolium-4,5-dicarboxylic acid] was heated in 5 ml of benzenesulphonic acid ethyl ether at 120° C. for 3 hours. The reaction mixture was diluted by an appropriate solvent, such as water-free diethyl ether or water-free acetone, and the residue was filtered. The yield was 1.4 g (74.2%). The melting point was 108-111° C. (n-butanol n-heptane). The results of elemental analysis and NMR spectroscopy are set out in Tables 1 and 2 respectively.

### EXAMPLE 4

### 1-methyl-3-ethyl-4,5-bis(N-methylcarbamoyl)-imidazolium chloride (4)

1 g of 1-methyl-3-ethyl-4,5-bis(N-methylcarbamoyl)-imidazolium benzenesulfonate (2) was dissolved in 20 ml of distilled water, and the solution was then heated with the ion-exchange resin Amberlite IRA-410 (Cl⁻-form, 40 ml, eluent water). 600 ml of eluent was collected, and the water was evaporated under vacuum. The residue was dried under vacuum over P₂O₅ at room temperature for 12-18 hours. Compound (4) was obtained as a white hygroscopic powder. The yield was 0.68 g (98%). The melting point was 192-194° C. The results of elemental analysis and NMR spectroscopy are set out in Tables 1 and 2 respectively.

### EXAMPLE 5

### 1-methyl-3-ethyl-4,5-bis(N-methylcarbamoyl)-imidazolium benzoate (5)

1 g of 1-methyl-3-ethyl-4,5-bis(N-methylcarbamoyl)-imidazolium benzenesulfonate (2) was dissolved in 20 ml of distilled water, and the solution was then heated with the ion-exchange resin Amberlite IRA-410 (C₆H₅COO⁻-form, 40 ml, water eluent). 600 ml of eluent was collected, and the water was evaporated under vacuum. The residue was dried under vacuum above P₂O₅ at room temperature for 12-18 hours. Compound (5) was obtained as a white hygroscopic powder. The yield was 0.89 g (97%). The melting point was 140-145° C. The results of elemental analysis and NMR spectroscopy are set out in Tables 1 and 2 respectively.

### EXAMPLE 6

### 1-methyl-3-ethyl-4,5-bis(N-methylcarbamoyl)-imidazolium salicylate (6)

6 g of 1-methyl-3-ethyl-4,5-bis(N-methylcarbamoyl)-imidazolium benzenesulfonate (2) were dissolved in 200 ml of distilled water. The solution was then heated with ion-exchange resin Amberlite IRA-410 10 (OH⁻-form), and the eluent was collected in a 2-litre beaker containing 3.5 g (0.024 mole) of salicylic acid per 15 ml of water. The volume of the eluent was 1.5 litres. The undissolved salicylic acid was filtered, and the dihydrate of compound (6) was extracted by lyophilization to give a light yellow hygroscopic powder. The yield was 5.85 g (98%). The melting point was 109-110° C. The results of elemental analysis and NMR spectroscopy are set out in Tables 1 and 2 respectively.

### EXAMPLE 7

### 1-methyl-3-ethyl-4,5-bis(N-methylcarbamoyl)-imidazolium 2,4-dihydroxybenzoate (7)

6 g (0.016 mole) of 1-methyl-3-ethyl-4,5-bis(N-methylcarbamoyl)-imidazolium benzenesulfonate (2) were dissolved in 200 ml of distilled water. The solution was then heated with ion-exchange resin Amberlite IRA-410 10 (OH⁻-form), and the eluent was collected in a 2-litre beaker containing 3.85 g (0.025 mole) of 2.5 dihydroxybenzoic acid per 15 ml of water. The volume of the eluent was 1.5 litres. The undissolved acid was filtered, and compound (7) was extracted by lyophilization to give a light yellow hygroscopic powder. The yield was 5.76 g (97%). The melting point was 168-170° C. The results of elemental analysis and NMR spectroscopy are set out in Tables 1 and 2 respectively.

### EXAMPLE 8

### Alternative synthesis of Compounds (6) and (7)

Compounds (6) and (7) can also be obtained by heating the solution of compound (2) with ion-exchange resin containing salicylic or 2.5 dihydroxybenzoic acid, similarly to the method used to produce compounds (4) and (5), with the final products being isolated by lyophilization.

TABLE 1
Results of elemental analysis of compounds I-VII

| Com- | Molecular | Found | | | Calculated | | |
|---|---|---|---|---|---|---|---|
| pound | formula | C | H | N | C | H | N |
| 1 | C₁₅H₂₀N₄O₅S | 48.95 | 5.60 | 14.63 | 48.90 | 5.47 | 15.21 |
| | | 48.48 | 5.47 | 14.87 | | | |
| 2 | C₁₆H₂₂N₄O₅S | 50.81 | 5.84 | 14.41 | 50.25 | 5.80 | 14.65 |
| | | 50.81 | 6.13 | 14.65 | | | |
| 3 | C₁₇H₂₄N₄O₅S | 51.18 | 6.07 | 14.13 | 51.50 | 6.10 | 14.13 |
| | | 51.23 | 6.11 | 14.33 | | | |
| 4 | C₁₀H₁₇N₄O₂Cl | 46.13 | 6.57 | 21.47 | 46.07 | 6.57 | 21.29 |
| | | 46.35 | 6.31 | 21.06 | | | |
| 5 | C₁₇H₂₂N₄O₄ | 58.75 | 6.39 | 15.94 | 58.93 | 6.41 | 16.18 |
| | | 59.08 | 6.77 | 16.09 | | | |
| 6 | C₁₇H₂₂N₄O₅ 2H₂O | 51.71 | 6.40 | 13.94 | 51.25 | 6.58 | 14.06 |
| | | 51.55 | 6.35 | 14.21 | | | |
| 7 | C₁₇H₂₂N₄O₆ | | | 14.93 | | | 14.81 |
| | | | | 15.01 | | | |

TABLE 2
Results of NMR analysis of compounds 1-7(DMSO-d6)

| Com- | |
|---|---|
| pound | δ, ppm. |
| 1 | 2.78 s[3H, (NH)—CH₃], 2.80 s[3H, (NH)—CH₃], 3.87 s[6H, |
| | N1(3)—CH₃], 7.31-7.60[5H, arom H], 8.77-8.78[2H, NH], |
| | 9.26 s[1H, (C2)—H] |
| 2 | 1.37 t[3H, CH₂—CH₃, J=7Hz], |
| | 2.79 s[3H, NH—CH₃], 2.80 s[3H, (NH)—CH₃], 3.87 s[3H, |
| | N1—CH₃], 4.28 q[2H, CH₂, J=7Hz], 7.32-7.60[5H, arom H], |
| | 8.73-8.82[2H, NH], |
| | 9.35 s[1H, (C2)—H] |
| 3 | 1.38 t[6H, 2(CH₂)—CH₃, J=7Hz], 2.80 s[6H, 2(NH)—CH₃], |
| | 4.30 q[2H, 2(CH₃)—CH₂, J=7Hz], 7.31-7.59[5H, arom H], |
| | 8.80[2H, 2NH], |
| | 9.41 s[1H, (C2)—H] |
| 4 | 1.38 t[3H, CH₂—CH₃, J=7Hz], 2.78-2.79[6H, (NH)—CH₃], |
| | 3.90 s[3H, N1—CH₃], 4.30 q[2H, CH₂, J=7Hz], 9.35 s[1H, |
| | NH], |
| | 9.42 s[1H, NH], 9.64 s[1H, (C2)—H] |
| 5 | 1.31 t[3H, CH₂—CH₃, J=7Hz], 2.81 s[6H, 2 NH—CH₃], |
| | 3.79 s[3H, N1—CH₃], 4.20 q[2H, CH₂, J=7Hz], |
| | 7.29-7.78 m[5H, arom H], 9.42-10.1[3H, 2 NH, (C2)—H] |
| 6 | 1.37 t[3H, CH₂—CH₃, J=7Hz], 2.81 s[6H, 2 NH—CH₃], |
| | 3.87 s[3H, N1—CH₃], 4.30 q[2H, CH₂, J=7Hz], |
| | 6.63-7.71[4H, arom H], 8.94 s[1H, NH], 9.00 s[1H, NH], 9.40 |
| | s[1H, (C2)—H] |
| 7 | 1.37 t[3H, CH₂—CH₃, J=7Hz], 2.80 s[6H, 2 NH—CH₃], |
| | 3.86 s[3H, N1—CH₃], 4.28 q[2H, CH₂, J=7Hz], |
| | 6.45-7.15[3H, arom H], |
| | 8.5 s[1H], 9.02-9.08 s[2H, 2NH], 9.40 s[1H, (C2)—H] |

### EXAMPLE 9

### Effect of 1,3-dialkyl-4,5-bis (N-methylcarbamoyl)imidazolium salts on acute Con A-induced inflammation

1,3-dialkyl-4,5-bis (N-methylcarbamoyl)imidazolium salts at doses of 10 and 50 mg/kg were injected intra-peritoneally into male CBA mice, weighing between 18 and 20 g, one hour before the injection of concanavalin A (con A). Saline solution was similarly administered to the control animals. There were 10 mice in each group. After one hour, con A at a dose of 100 mkg/20 g of body weight was administered by sub-plantar injection into the plantar aponeurosis of the left paws of mice of both the experimental and control groups. The same amount of saline splution was injected into the contralateral paws. After one hour, the mice were killed and the experimental and control paws were weighed to assess the reaction index of the inflammation in the ankle-joint (Liubimov et al, 1999).

We found that the maximum swelling of ankle joints was observed 1 hour after the sub-plantar administration of Con A. Intraperitoneal administration of 1,3-dialkyl-4,5-bis(N-methylcarbamoyl)-imidazolium salts at a dose of 10 to 50 mg/kg one hour before the induction of the oedema reduced the intensity of the inflammatory process. These results are summarised in Table 3.

TABLE 3
Effect of 1,3-dialkyl-4,5-bis (N-methylcarbamoyl)-imidazolium
salts on acute Con A-induced inflammation in mice

| Medication | Reaction index (%) |
|---|---|
| Saline solution | 20.2 ± 3.8 |
| Compound (1)10 mg/kg | 14.6 ± 3.5 |
| Compound (1)50 mg/kg | 5.3 ± 2.0** |
| Compound (2)10 mg/kg | 10.4 ± 2.0* |
| Compound (2)50 mg/kg | 8.4 ± 0.9* |
| Compound (3)10 mg/kg | 9.5 ± 2.5* |
| Compound (3)50 mg/kg | 9.5 ± 2.0* |
| Compound (4)10 mg/kg | 14.1 ± 2.0 |
| Compound (4)50 mg/kg | 12.4 ± 0.4* |
| Compound (5)50 mg/kg | 7.2 ± 1.8* |

*significant difference between experimental group and control (Con A) - P < 0.001;
**difference between the effect of compound (1)(50 mg/kg) and controls.

### EXAMPLE 10

### Comparison of the anti-inflammatory effect of Compound 2 and Compound 7

The anti-inflammatory effect of Compound 2 and Compound 7 was compared in male mice weighing 18-22 g, using the model described in Example 9. The ED₅₀, i.e. the dose reducing paw ooedema by 50% one hour after injection of Concanavalin A, was calculated. The results are shown in Table 4, and indicate that ED₅₀ for Compound 2 was 33 mg/kg, and ED₅₀ for Compound 7 was 52 mg/kg.

TABLE 4
Comparison of anti-inflammatory effect of compound 2 and compound 7

| | | |
|---|---|---|
| | Reaction | Paw oedema |
| Group of animals | Index | reduction |
| n = 10 | (%) | (%) |

| | | |
|---|---|---|
| Saline solution + Con A | 16.0 | 0 |
| Compound 2 10 mg/kg + Con A | 11.1 ± 3.5 | 33 |
| Compound 2 19 mg/kg + Con A | 5.4 ± 2.0 | 66 |
| (0.005M) | | |
| Compound 2 38 mg/kg + Con A | 7.7 ± 1.8 | 52 |
| (0.01M) | | |
| Compound 2 57 mg/kg + Con A | 5.3 ± 2.0 | 66 |
| (0.015M) | | |
| Compound 2 76 mg/kg + Con A | 3.3 ± 2.0 | 77 |
| (0.02M) | | |
| Compound 7 16.4 mg/kg + Con A | 6.73 ± 2.2 | 60 |
| (0.005M) | | |
| Compound 7 32 mg/kg + Con A | 9.93 ± 2.8 | 13 |
| (0.01M) | | |
| Compound 7 49 mg/kg + Con A | 7.2 ± 1.8 | 45 |
| (0.015M) | | |
| Compound 7 65.6 mg/kg + Con A | 4.9 ± 1.7 | 70 |
| (0.02M) | | |

### EXAMPLE 11

### Effect of Compound (2) on carrageenin-induced inflammation in intact and adrenalectomized animals

An acute inflammatory oedema was induced in intact and adrenalectomized male rats weighing 180-200 g by sub-plantar injection of 0.1 ml of 1% carrageenin solution into the left paw (Winter et al., 1962). The same volume of saline solution was injected into the contralateral paw. Compound (2) at doses of 50 and 100 mg/kg and ibuprofen as a comparative medication at a dose of 48 mg/kg, corresponding to the literature ED₅₀ value, were administered orally into the stomach via an intra-gastric tube 4 times, daily each day for 3 days and one hour before administration of carrageenin. There were 10 rats in each group.

The degree of carrageenin-induced inflammation of the ankle joints was assessed at 3 hours after the induction of the inflammation, on the basis of the change in weight of the experimental and control paws (reaction index). The results are summarised in Table 5, and show that compound 2 reproducibly reduced the inflammation in both intact and adrenalectomized animals.

TABLE 5
Effect of prolonged administration of compound (1) on the
stage of acute inflammation in rats caused by carrageenin

| | Reaction index | Inhibition of |
|---|---|---|
| Group | (%) | oedema(%) |
| Intact animals + saline | 43.04 ± 3.58 | 0 |
| solution + carrageenin | | |
| Intact animals + compound | 29.96 ± 2.30*,** | 30.41 |
| (2)50 mg/kg 4 days + carrageenin | | |
| Adrenalectomized animals + compound | 31.04 ± 2.49*,** | 27.88 |
| (2)50 mg/kg 4 | | |
| days + carrageenin | | |
| Intact animals + compound | 22.63 ± 2.86*,** | 47.43 |
| (2)100 mg/kg 4 days + carrageenin | | |
| Adrenalectomized animals + compound | 25.63 ± 2.86*,** | 44.43 |
| (2)100 mg/kg 4 | | |
| days + carrrageenin | | |
| Ibuprofen 48 mg/kg 4 days + | 15.88 ± 1.88* | 63.39 |
| carrageenin | | |
| Adrenalectomized animals + ibuprofen | 18.12 ± 1.82* | 57.90 |
| 48 mg/kg orally | | |
| 4 days + carrageenin | | |

*significant difference between the experiment groups and controls, P < 0:001;
**significant difference between the effect of compound (I) and controls.

### EXAMPLE 12

### Effect of Compound (2) on bradykinin-induced inflammation in intact animals

Acute inflammation was induced by sub-plantar injection of 0.1 ml of 0.01% of bradykinin in saline solution into the left paws of male rats weighing 180-200 g. The acuteness of the inflammation was assessed 30 minutes after injection of bradykinin by the change in the paw volume. The anti-inflammatory effect, assessed by the reduction of swelling, was expressed as a percentage of the initial paw weight compared to controls. The test substances were administered orally via an intra-gastric tube for three days and at one hour before administration of bradykinin. The following agents were used for comparison:
Control: saline alone
Butadione: 60 mg/kg: (anti-inflammatory agent)
Paramidine: 50 mg/kg (pyridinole carbamate; Buclome (Takeda); specific anti-bradykinin agent).
   These were administered orally to rats for 3 days and at one hour before the injection of the agent. There were 12 rats in each group. The doses of butadione and paramidine correspond to literature ED₅₀ values.

Compound (2) at a dose of 50 mg/kg resulted in an obvious lessening of the swelling reaction caused by bradykinin. This effect was equal to the effect of the specific anti-bradykinin activity of paramidin, and similar to that of butadione. The results are summarised in Table 6.

TABLE 6
Effect of compound (2) on acute bradykinin-induced
inflammation in rats

| | Paw volume | | | |
|---|---|---|---|---|
| | (ml of water reduction) | | | |
| | | 30 minutes | Increase | |
| | | after | in paw | Swelling |
| | | administration | volume | inhibition |
| Medication | initial | of bradykinin | (ml) | (%) |
| Bradykinin | 1.23 ± 0.043 | 1.92 ± 0.070 | 0.69 | 0 |
| Compound | 1.39 ± 0.040 | 1.80 ± 0.060 | 0.41 | 40.58 |
| (2) | | | | |
| 20 mg/kg | | | | |
| Compound | 1.35 ± 0.038 | 1.69 ± 0.060* | 0.34 | 50.72 |
| (2) | | | | |
| 50 mg/kg | | | | |
| Paramidine | 1.27 ± 0.038 | 1.61 ± 0.051* | 0.34 | 50.72 |
| 50 mg/kg | | | | |
| Butadione | 1.33 ± 0.061 | 1.60 ± 0.07* | 0.27 | 60.87 |
| 60 mg/kg | | | | |

*significant difference between butadione, paramidine, compound (2) and controls - P < 0:001.

### EXAMPLE 13

### Effect of compound (2) on chronic proliferative inflammation (granuloma)

We investigated the ability of compound (2) to reduce the formation of granulation tissue in the inflammatory focus which develops at the site at which a non-sterile cotton-wool roll weighing 40 mg is implanted subcutaneously (Swingle and Shideman, 1972). Compound (2) was administered orally to male rats weighing 130-150 g at a dose of 100 mg/kg for 6 days before the cotton-wool roll was implanted, and then for 6 days at the same dose while the granuloma developed. Anti-inflammatory agents, such as ibuprofen and butadione, were used for comparison, using the same procedure. There were 10 rats in each group.

After 12 days the experiment was terminated, and the granulomas were excised, weighed, and the wet weight of the granulation tissue was measured. The dry mass of the granuloma was measured after drying for 24 hours at 70° C., and the exudative and proliferative stages of the granulomas were calculated. Statistical analysis was performed using the Student's parametric test. The results are summarised in Table 7, and show that compound (2) is able to inhibit granuloma formation to an extent similar to that of conventional anti-inflammatory agents.

TABLE 7
Effect of compound (2) on the development of
experimental granuloma in rats

Granuloma weight (mg)

| Group | Fresh weight | Dry weight |
|---|---|---|
| Controls | 1375.8 ± 61.85 | 249.6 ± 13.27 |
| (0.5 ml starch) | Exudative stage | Proliferative |
| | 1126.2 | stages |
| | | 209.6 |
| Compound (2) | 956.29 ± 115.5* | 180.71 ± 14.16* |
| (100 mg/kg) | Exudative stage | Proliferative |
| | 775.58 | stages |
| | | 140.71 |
| Ibuprofen (48 mg/kg) | 814.0 ± 63.72** | 143.2 ± 9.83** |
| | Exudative stage | Proliferative |
| | 670.8 | stages |
| | | 103.2 |
| Butadione (60 mg/kg | 828.0 ± 76.68** | 145.4 ± 7.45** |
| | Exudative stage | Proliferative |
| | 682.6 | stages |
| | | 105.4 |

*significant difference between the second experimental group and controls (P < 0.01);
**significant difference between the third experimental group and controls; significant difference between the fourth group and controls (P < 0.001).

### EXAMPLE 14

### Effect of topically-applied Compound 2 on wound healing

To heal successfully, wounds should be treated with regard to specific features of the various stages of healing process of the wound (Kuzin et al, 1981). The wound-healing effect of salts of N-substituted 1,3-dialkyl-4,5-bis(carbamoyl)imidazolium derivatives was studied in a model of aseptic full-thickness skin wounds in rats (Zapadnyuk et al, 1983). The effect of 10% 1-ethyl-3-methyl-4,5-bis(N-methylcarbamoyl)imidazolium benzensulfonate in a lanolin ointment base was compared to that of Solcoseryl gel and Spasatel balm.

Statistical analysis of the data was performed using standard methods involving the Student's parametric test, based on the programme developed in the Department of Neuropharmacology of the Institute of Experimental Medicine of the Russian Academy of Medical Sciences. Healing of a full-thickness wound was assessed on the basis of a set of morphological criteria characterising the quality of the regeneration process.

Full-thickness wounds are wounds in which all three layers of the skin, ie. the epidermis, dermis and the subdermis are damaged. Reparative and regenerative tissue processes affect each of the three layers. Healing occurs by so-called “primary intention” if the wound is aseptic or is thoroughly cleaned early in the process; regenerative processes in the epithelium occur in tandem and are complete by the fifteenth day, with formation of granulation tissue and proliferation and differentiation of regenerative tissues. “Secondary intention” healing is healing in the presence of infection or after loss of a large area of tissue, for example following major trauma or burn. Secondary intention healing is always accompanied by scar formation.

Aseptic full-thickness skin wounds were created in rats under light ether anaesthesia. At a site on the animal's back where the animal could not lick the wound, the skin was shaved, and a circular piece of skin approximately 2.5 cm² area was excised with scissors. Following recovery from anaesthesia the animals were kept in separate cages, with normal food and water ad libitum. A total of 100 rats weighing from 200 to 250 g were used, with 20 rats in each group. The groups were as follows:

1. Untreated control (self-healing)

2. Placebo control (lanolin ointment base only)

3. Test (10% Compound 2 in lanolin ointment base)

4. Comparative Group A: Solcoseryl gel (Solco, Switzerland)

5. Comparative Group B: Spasatel balm (AOE Effect, Ukraine).

Treatment was performed 24 hours after the wounding by applying 200 g of ointment once a day in a proportion of 80 mg of ointment per 1 cm² of the wound area (8 mg of substance per 1 cm²) and then daily until healing was complete.

Solcoseryl is widely used in Russia as a wound healing medication; this composition contains deproteinised haemodialysate of male calf blood. It has been shown to induce healing by secondary intention. Spasatel is a multi-component balsam containing sea buckthorn oil, naphthalene, beeswax, essential oils derived from various plants, and vitamins. This composition induces healing by secondary intention, and often leaves residual scarring.

Animals were examined at 5, 10, 15 and 20 days for the following parameters; wound size and wound healing time; weight gain, peripheral blood indicators (total leucocyte count, differential leucocyte count, and erythrocyte sedimentation rate); and biochemical serum indicators (total protein, albumin, globulin, and globulin fractions); and macroscopic and microscopic assessment of wounds. The average complete wound healing period was 16.8±1.9 days for Compound 2, compared to 23.5±2.2 days for Spasatel and 24.0±2.4 days for Solcoseryl. Thus the compound of the invention was significantly more effective than either of the prior art agents.

For measurement of wound size, a sterile object plate was laid over the wound, and its contour was outlined with a marker. The wounds were photographed, the wound outlines were scanned, and their area was calculated using an in-house computer program.

The results of the re-measurement of the wound area illustrate the dynamics of regeneration processes of both the epithelium covering the wound and the underlying granulation tissue. The average area figures in control and test groups were compared. A comparative analysis of the wound healing dynamics indicated that the wound area for the animals treated with 10% Compound 2 ointment was 0.4% on the fifteenth day, compared to 10.4% for Solcoseryl, 11% for Spasatel and 24.1% for placebo.

The results are presented in Tables 8 to 10. The most marked wound healing effect was observed with Compound 2 ointment; the difference from the controls and from the comparison groups was significant (p<0.05).

Healing in response to treatment with Compound 2 takes place by primary intention, in which the wound is rapidly and fully cleaned, and regeneration processes in the epithelium-connective tissue system, which are synchronised and completed by the 15th day, proceed without any evidence of pathological regeneration or differentiation of connective tissue.

TABLE 8
Duration of wound healing

| | | Epithelialization |
|---|---|---|
| Nos. | Group of animals | (duration in days) |
| 1. | Control (self-healing) | 29.2 ± 1.1 |
| 2. | Placebo¹ | 24.5 ± 1.1* |
| 3. | Compound 2² (10% | 16.8 ± 1.9*^{.}** |
| | ointment) | |
| 4. | Solcoseryl gel | 24.0 ± 2.4* |
| 5. | Spasatel balm | 23.5 ± 2.3* |

¹ointment base - lanolin
²1-methyl-3-ethyl-4,5-bis(N-methylcarbamoyl)imidazolium benzensulfonate
*significant difference compared to control (P < 0.05)
**significant difference compared to placebo (P < 0.05)

TABLE 9
WOUND HEALING DYNAMICS
Wound area (% of area before treatment)

| | | Before | | | | |
|---|---|---|---|---|---|---|
| Animal Group | | treatment | 5^{th} day | 10^{th} day | 15^{th} day | 20^{th} day |
| 1. | Placebo¹ | 100.0 | 106.9 | 43.8 | 24.1 | 11.6 |
| 2. | Test compound² | 100.0 | 84.0 | 8.1 | 0.4 | 0.1 |
| | (10% ointment) | | | | | |
| 3. | Solcoseryl | 100.0 | 89.2 | 22.6 | 10.4 | 3.8 |
| 4. | Spasatel | 100.0 | 89.6 | 23.8 | 11.0 | 5.8 |

¹lanolin ointment base
²1-ethyl-3-methyl-4,5-bis (N-methylcarbamoyl)imidazolium benzenesulfonate (compound 2)

TABLE 10
WOUND HEALING PERIOD

| | Animal group | Epithelialization period |
|---|---|---|
| 1. | Control | 29.2 ± 1.1 |
| | (untreated) | |
| 2. | Placebo¹ | 24.5 ± 1.1 |
| 3. | Test compound² | 16.8 ± 1.9** |
| | (10% ointment) | |
| 4. | Solcoseryl | 24.0 ± 2.4 |
| 5. | Spasatel | 23.5 ± 2.2 |

*differences are significant as compared to the control group (self-healing), P < 0.05
**differences are significant as compared to the control group (placebo), P < 0.05
¹lanolin ointment base
²1-ethyl-3-methyl-4,5-bis (N-methylcarbamoyl)imidazolium benzenesulfonate (compound 2)

Body weight was checked once a week before feeding the animals. The results are presented in table 11. Daily observations indicated that a statistically significant weight gain was observed only in the animals who were treated with Compound 2. On the twentieth day, in the test group this indicator was 19%, whereas in the control (untreated and placebo) groups it was 10-12%. These results confirm the positive effect of the test compound on wound repair and regeneration processes.

TABLE 11
Rat body weight changes during full-thickness wound treatment

| | | Before | | | | | | | | | |
|---|---|---|---|---|---|---|---|---|---|---|---|
| | | treatment | | 5^{th} day | | 10^{th} day | | 15^{th} day | | 20^{th} day | |
| Animal Group | | g | % | g | % | g | % | g | % | g | % |
| 1. | Control | 272 ± 12.6 | 100 | 279 ± 10.5 | 102.6 | 279 ± 8.4 | 102.6 | 294 ± 11.6 | 108.1 | 301 ± 12.6 | 110.7 |
| | (untreated) | | | | | | | | | | |
| 2. | Placebo¹ | 229 ± 13.7 | 100 | 240 ± 16.8 | 104.8 | 245 ± 12.6 | 107.0 | 252.5 ± 12.6 | 110.3 | 257.5 ± 12.6 | 112.4 |
| 3. | Test compound² | 226 ± 8.4 | 100 | 236 ± 8.4 | 104.4 | 246 ± 1.1 | 108.8 | 253.3 ± 2.1 | 112.1 | 268.0 ± 4.2 | 118.6 |
| | (10% ointment) | | | | | | | | | | |
| 4. | Solcoseryl | 235 ± 6.3 | 100 | 250 ± 6.3 | 106.4 | 264 ± 8.4 | 112.3 | 272 ± 10.5 | 115.7 | 279.0 ± 8.4 | 118.7 |
| 5. | Spasatel | 230 ± 8.4 | 100 | 244 ± 10.5 | 106.1 | 250 ± 10.5 | 108.7 | 255 ± 12.6 | 110.9 | 258.0 ± 12.6 | 112.2 |

¹lanolin ointment base
²1-ethyl-3-methyl-4,5-bis (N-methylcarbamoyl)imidazolium benzenesulfonate (Compound 2)

Peripheral blood indicators (leucocytes, leucocyte formula, erythrocyte sedimentation rate) of the rats from the test and control groups were measured before the animals were wounded, and then checked on the fifth, tenth and fifteenth days of the experiment. The results of these experiments are presented in Tables 12, 13, and 14. No reproducible changes compared to the blood characteristics of the intact animals prior to wounding were identified from the haematological indicators of rats treated with Compound 2.

TABLE 12
Rat peripheral blood indicators during full-thickness skin wound treatment - day 5

| | | | | Leucocyte count (%) | | | | | |
|---|---|---|---|---|---|---|---|---|---|
| Animal | | | Leucocytes | Band | Segmented | | | | |
| group | | ESR mm/h | 10⁹/l | neutrophils | neutrophils | eosinophils | basophils | monocytes | lymphocytes |
| 1 | Intact | 2.3 ± 1.3 | 7.2 ± 1.3 | 1.3 ± 0.3 | 30.0 ± 5.1 | 1.7 ± 0.3 | 0 | 3.7 ± 0.7 | 63.3 ± 6.2 |

5 days

| | | | | | | | | | |
|---|---|---|---|---|---|---|---|---|---|
| 2 | Control | 1.8 ± 0.4 | 5.3 ± 0.2 | 1.2 ± 0.2 | 43.2 ± 3.0 | 2.0 ± 0.4 | 0.4 ± 0.2 | 3.6 ± 0.9 | 49.6 ± 3.7 |
| | (untreated) | | | | | | | | |
| 3 | Placebo¹ | 1.8 ± 0.4 | 5.5 ± 0.3 | 1.2 ± 0.2 | 37.8 ± 3.9 | 2.8 ± 0.6 | 0.4 ± 0.2 | 5.0 ± 1.1 | 52.8 ± 3.2 |
| 4 | Test | 2.2 ± 0.6 | 6.6 ± 0.9 | 1.4 ± 0.4 | 35.2 ± 4.4 | 2.6 ± 0.6 | 0 | 8.2 ± 0.8 | 52.6 ± 4.5 |
| | compound² | | | | | | | | |
| | (10% | | | | | | | | |
| | ointment) | | | | | | | | |
| 5 | Solcoseryl | 1.0 ± 0 | 5.8 ± 0.4 | 1.2 ± 0.2 | 40.2 ± 2.5 | 3.2 ± 0.7 | 0.4 ± 0.2 | 4.4 ± 0.8 | 51.0 ± 3.3 |
| 6 | Spasatel | 1.8 ± 0.4 | 5.2 ± 0.4 | 1.0 ± 0 | 39.4 ± 3.0 | 3.6 ± 0.5 | 0 | 5.0 ± 1.2 | 50.8 ± 2.7 |

¹lanolin ointment base
²1-ethyl-3-methyl-4,5-bis (N-methylcarbamoyl)imidazolium benzenesulfonate (Compound 2)

TABLE 13
Rat peripheral blood indicators during full-thickness skin wound treatment - day 10

| | | | Leucocyte count (%) | | | | | |
|---|---|---|---|---|---|---|---|---|
| Animal | | Leucocytes | Band | Segmented | | | | |
| group | ESR mm/h | 10⁹/l | neutrophils | neutrophils | eosinophils | basophils | monocytes | lymphocytes |

10 days

| | | | | | | | | | |
|---|---|---|---|---|---|---|---|---|---|
| 1 | Control | 1.2 ± 0.2 | 8.6 ± 1.0 | 3.0 ± 0.5 | 23.5 ± 5.2 | 2.7 ± 0.5 | 0.4 ± 0.2 | 3.4 ± 0.6 | 67.0 ± 5.9 |
| | (untreated) | | | | | | | | |
| 2 | Placebo¹ | 1.3 ± 0.3 | 7.2 ± 1.1 | 1.3 ± 0.3 | 39.3 ± 1.3 | 3.3 ± 1.0 | 0 | 6.0 ± 0.7 | 50.3 ± 1.7 |
| 3 | Test | 1.0 ± 0 | 7.9 ± 0.6 | 1.2 ± 0.2 | 29.6 ± 2.2 | 2.4 ± 0.5 | 0.6 ± 0.2 | 2.4 ± 0.5 | 63.8 ± 1.9 |
| | compound² | | | | | | | | |
| | (10% | | | | | | | | |
| | ointment) | | | | | | | | |
| 4 | Solcoseryl | 1.6 ± 0.4 | 9.2 ± 0.8 | 1.8 ± 0.4 | 29.6 ± 2.5 | 3.2 ± 0.6 | 0.2 ± 0.2 | 2.8 ± 0.6 | 62.4 ± 2.5 |
| 5 | Spasatel | 1.2 ± 0.2 | 9.4 ± 0.8 | 1.4 ± 0.2 | 40.0 ± 3.0 | 2.0 ± 0.6 | 0.2 ± 0.2 | 1.8 ± 0.4 | 54.6 ± 2.3 |

¹lanolin ointment base
²1-ethyl-3-methyl-4,5-bis (N-methylcarbamoyl)imidazolium benzenesulfonate (Compound 2)

TABLE 14
RAT PERIPHERAL BLOOD INDICATORS DURING FULL-THICKNESS SKIN WOUND TREATMENT - DAY 15

| | European | | | | | | | |
|---|---|---|---|---|---|---|---|---|
| | Search | | Leucocyte count (%) | | | | | |
| Animal | Report | Leucocytes | Band | Segmented | | | | |
| group | mm/h | 10⁹/1 | neutrophils | neutrophils | eosinophils | basophils | monocytes | lymphocytes |

15 days

| | | | | | | | | | |
|---|---|---|---|---|---|---|---|---|---|
| 1 | Control | 1.8 ± 0.4 | 10.1 ± 2.3 | 1.8 ± 0.4 | 35.4 ± 5.7 | 3.6 ± 1.7 | 0.2 ± 0.2 | 5.4 ± 1.7 | 53.8 ± 5.8 |
| | (untreated) | | | | | | | | |
| 2 | Placebo¹ | 1.4 ± 0.2 | 9.4 ± 1.4 | 1.4 ± 0.2 | 29.4 ± 2.6 | 2.2 ± 0.6 | 0.4 ± 0.2 | 4.8 ± 0.6 | 62.0 ± 2.3 |
| 3 | Test | 1.4 ± 0.2 | 11.1 ± 1.6 | 1.2 ± 0.2 | 21.0 ± 3.1 | 2.8 ± 0.2 | 0.4 ± 0.2 | 4.0 ± 0.7 | 70.6 ± 3.4 |
| | compound² | | | | | | | | |
| | (10% | | | | | | | | |
| | ointment) | | | | | | | | |
| 4 | Solcoseryl | 1.6 ± 0.2 | 7.8 ± 1.2 | 1.2 ± 0.2 | 31.8 ± 4.9 | 5.6 ± 1.3 | 0.4 ± 0.2 | 4.2 ± 0.9 | 56.0 ± 5.3 |
| 5 | Spasatel | 1.4 ± 0.2 | 6.6 ± 0.7 | 1.0 ± 0 | 34.0 ± 2.4 | 3.0 ± 0.9 | 0.6 ± 0.4 | 6.4 ± 1.2 | 55.0 ± 1.9 |

¹lanolin ointment base
²1-ethyl-3-methyl-4,5-bis (N-methylcarbamoyl)imidazolium benzenesulfonate (Compound 2)

Total protein in the blood of test and control animals was determined using the biuret method, and protein fractions (albumins and globulins: α₁, α₂, β and γ) were measured electrophoretically prior to the experiment and then on the fifth, tenth and fifteenth days. The data presented in tables 15, 16, and 17 show that on the fifth day all groups of animals demonstrated a decrease in albumin and increase in globulin fractions in their blood as a result of inflammation. These values returned to normal levels at days 10 and 15. These changes in the protein parameters reflect general patterns in response to skin damage.

TABLE 15
Total serum protein and protein fractions - day 5

| Animal | | Total | | | Globulins | | | | |
|---|---|---|---|---|---|---|---|---|---|
| Group | | Protein | Albumin % | Globulin % | á₁ | á₂ | â | ã | A/G |
| 1 | Intact | 65.57 ± 1.15 | 44.8 ± 0.7 | 55.2 ± 0.7 | 12.1 ± 1.4 | 9.0 ± 1.5 | 21.4 ± 1.99 | 12.7 ± 1.2 | 0.82 ± 0.02 |

5 days

| | | | | | | | | | |
|---|---|---|---|---|---|---|---|---|---|
| 2 | Control | 56.80 ± 1.72 | 39.2 ± 3.6 | 60.8 ± 3.6 | 19.4 ± 0.9 | 10.9 ± 1.1 | 16.5 ± 0.96 | 14.1 ± 3.1 | 0.67 ± 0.09 |
| | (untreated) | | | | | | | | |
| 3 | Placebo¹ | 59.34 ± 0.89 | 41.8 ± 1.4 | 58.2 ± 1.4 | 17.1 ± 1.5 | 13.3 ± 0.6 | 17.6 ± 2.3 | 10.1 ± 1.2 | 0.72 ± 0.04 |
| 4 | Test | 57.45 ± 0.64 | 37.2 ± 2.5 | 62.8 ± 2.5 | 16.8 ± 0.4 | 16.0 ± 1.5 | 18.3 ± 1.4 | 11.3 ± 0.7 | 0.60 ± 0.07 |
| | compound² | | | | | | | | |
| | (10% | | | | | | | | |
| | ointment) | | | | | | | | |
| 5 | Solcoseryl | 61.51 ± 2.18 | 37.3 ± 2.0 | 62.7 ± 2.0 | 17.0 ± 1.2 | 15.7 ± 1.5 | 21.5 ± 1.6 | 8.5 ± 1.3 | 0.60 ± 0.05 |
| 6 | Spasatel | 59.54 ± 2.05 | 41.0 ± 2.3 | 59.0 ± 2.3 | 19.6 ± 1.5 | 14.0 ± 1.3 | 16.7 ± 1.4 | 8.7 ± 0.5 | 0.71 ± 0.07 |

¹lanolin ointment base
²1-ethyl-3-methyl-4,5-bis (N-methylcarbamoyl)imidazolium benzenesulfonate (Compound 2)

TABLE 16
Total serum protein and protein fractions - day 10

| Animal | Total | | | Globulins | | | | |
|---|---|---|---|---|---|---|---|---|
| Group | Protein | Albumin % | Globulin % | á₁ | á₂ | â | ã | A/G |

10 days

| | | | | | | | | | |
|---|---|---|---|---|---|---|---|---|---|
| 1 | Control | 56.40 ± 0.98 | 43.5 ± 0.8 | 56.5 ± 0.8 | 11.9 ± 0.8 | 9.5 ± 0.9 | 21.0 ± 0.8 | 14.2 ± 0.6 | 0.77 ± 0.03 |
| | (untreated) | | | | | | | | |
| 2 | Placebo¹ | 66.88 ± 1.07 | 50.0 ± 1.9 | 50.0 ± 1.9 | 12.5 ± 0.3 | 8.3 ± 0.3 | 18.2 ± 1.6 | 11.0 ± 0.8 | 1.01 ± 0.08 |
| 3 | Test | 64.77 ± 1.44 | 42.9 ± 0.9 | 57.1 ± 0.9 | 13.2 ± 0.8 | 8.8 ± 0.5 | 21.7 ± 0.9 | 13.3 ± 0.6 | 0.75 ± 0.03 |
| | compound² | | | | | | | | |
| | (10% | | | | | | | | |
| | ointment) | | | | | | | | |
| 4 | Solcoseryl | 55.11 ± 0.76 | 41.5 ± 1.8 | 58.5 ± 1.8 | 15.6 ± 0.4 | 8.8 ± 0.8 | 20.0 ± 1.5 | 14.0 ± 0.7 | 0.72 ± 0.06 |
| 5 | Spasatel | 60.62 ± 0.87 | 43.4 ± 1.6 | 56.6 ± 1.6 | 16.0 ± 0.7 | 9.8 ± 0.6 | 18.4 ± 0.8 | 12.3 ± 0.8 | 0.77 ± 0.05 |

¹lanolin ointment base
²1-ethyl-3-methyl-4,5-bis (N-methylcarbamoyl)imidazolium benzenesulfonate (Compound 2)

TABLE 17
General protein and protein fractions of rats' blood serum

| Animal | Total | | | Globulins | | | | |
|---|---|---|---|---|---|---|---|---|
| Group | Protein | Albumin % | Globulin % | á₁ | á₂ | â | ã | A/G |

15 days

| | | | | | | | | | |
|---|---|---|---|---|---|---|---|---|---|
| 1 | Control | 58.93 ± 1.29 | 46.9 ± 1.7 | 53.1 ± 1.7 | 10.3 ± 1.0 | 10.2 ± 1.6 | 18.7 ± 0.4 | 13.9 ± 0.4 | 0.89 ± 0.06 |
| | (untreated) | | | | | | | | |
| 2 | Placebo¹ | 59.22 ± 1.65 | 48.2 ± 2.4 | 51.8 ± 2.4 | 10.1 ± 0.3 | 10.0 ± 1.1 | 17.3 ± 1.1 | 14.4 ± 0.6 | 0.94 ± 0.08 |
| 3 | Test | 59.06 ± 1.97 | 48.3 ± 1.7 | 51.7 ± 1.7 | 10.3 ± 0.9 | 7.9 ± 0.7 | 19.6 ± 1.5 | 13.8 ± 1.1 | 0.94 ± 0.06 |
| | compound² | | | | | | | | |
| | (10% | | | | | | | | |
| | ointment) | | | | | | | | |
| 4 | Solcoseryl | 57.93 ± 0.51 | 47.8 ± 0.6 | 52.2 ± 0.6 | 11.7 ± 0.9 | 7.6 ± 0.5 | 18.9 ± 0.9 | 13.9 ± 0.5 | 0.92 ± 0.02 |
| 5 | Spasatel | 63.24 ± 1.19 | 47.0 ± 2.4 | 53.0 ± 2.4 | 11.5 ± 0.9 | 8.1 ± 0.6 | 21.5 ± 1.3 | 11.9 ± 0.8 | 0.90 ± 0.08 |

¹lanolin ointment base
²1-ethyl-3-methyl-4,5-bis (N-methylcarbamoyl)imidazolium benzenesulfonate (Compound 2)

When medications are applied to the skin, physicochemical properties such as lipophilicity affect the rate at which the medication is liberated from the composition and absorbed into the skin (Scheuplein R. J, 1965). Our results demonstrate that, as a result of their low lipophilicity, the salts of N-substituted 1,3-dialkyl-4,5-biscarbamoylimidazole derivatives penetrate through biological membranes poorly, thus helping to prolong contact with the damaged area and increasing their efficiency during local application. Therefore these agents are suitable for local application, for example as an ointment, cream or gel.

### EXAMPLE 15

### Morphological assessment of the wound healing stages

An important indicator of wound healing is the morphology and epithelialization of the wound surface (Sarkisov et al, 1981). Samples were taken from experimental animals on the fifth, tenth and fifteenth days of the study described in Example 13, which roughly correspond to the three wound healing stages: the first traumatic inflammation stage is complete by the fifth day, the granulation tissue development stage is complete by the tenth day, and cicatrisation and epithelialization take place from the fifteenth to twentieth days. The results are illustrated in FIG. 1.

Day 5

a) Untreated control. The wounds were of significant size, covered by thick pellicles or scabs consisting of necrotic mass, fibrin, and leucocytes, with colonies of bacteria. Only 20% of animals cleaned the wound from the pellicle at individual spots; in the remaining cases the pellicle was closely connected to the wound surface, which was diffusely infiltrated by leucocytes. The granulation tissue on the bottom of the wound featured maturing and irregularly located vessels and fibres. There was productive inflammation and oedema in the areas adjacent to the wound. At the initial stages insignificant epithelialization is found at the edges of the wound. The degenerate wedge-shaped epithelialization layer has no distinct differentiation features.

b) Placebo. The wound was considerable in size, closely connected to the surface under the pellicle. There was intensive leucocyte infiltration of surface areas of the wound bottom, with the pellicle melting at the boundary. The border between the granulation tissue of the wound bottom and adjacent skin and hypoderma was indistinct, with weakly maturing features of the granulation tissue. 60% of the animals had insignificant epithelial regeneration at the edges of the wound, without distinct differentiation.

c) Compound 2

The wound was considerable in size, with focal removal of the pellicle, whose thickness was variable but often negligible. The surface was free of pellicle at the periphery of the wound, where regeneration of the epithelium was observed in all the animals. There was a wedge-shaped layer of regenerating epithelium of various sizes, with distinct signs of differentiation of flat cells. At the bottom of the wound, granulation tissue matured with inflammatory infiltration, which was more pronounced on surface sections. The boundaries between the bottom and the side sections of the wound were indistinguishable, due to oedema and inflammation.

Day 10

a) Untreated Control group. All of the wounds were covered with a thick pellicle consisting of necrotic mass, fibrin, leucocytes with colonies of bacteria. There were no signs of cleaning, and the pellicle was tightly bound to the bottom of the wound. On the bottom, there was maturing granulation tissue with a large number of newly formed vessels, irregular, chaotically-arranged collagen fibres, and pronounced leucocyte infiltration. There was diffuse inflammatory infiltration of the connective tissue, which is more intensive on the surface areas. In 60% of the cases, there were areas of purulent fusion of the connective tissue. Productive diffusive inflammation resulting in sclerosis was observed in the cellular tissue adjacent to the bottom and in the connective tissue. The inflammation was not limited to the bottom of the wound, and spread to the adjacent areas of the dermis. There was weak initial epithelial regeneration, with limited wedge-shaped regenerating areas at the edges of the wound and no distinct signs of differentiation.

b) Placebo. The cleaning of the wound surface was negligibble, and leucocytes were prevalent on the pellicle. In the areas without signs of cleaning, there was intensive leucocyte infiltration at the bottom of the maturing connective tissue. There were areas with a tendency towards cicatrisation of the connective tissue. The wound bottom did not have clear boundaries: the inflammation was spread over the adjacent dermis and fatty cellular tissue. Epithelial regeneration was virtually absent, and could be observed-only at one or two edges in 60% of the animals.

c) Compound 2

The surface of the wound showed irregular remains of the pellicle, represented by fibrin, erythrocytes and lysing leucocytes. Connective tissue with correct orientation of collagen fibres and moderate cell infiltration predominated at the bottom of the wound. The boundaries of the regenerating connective tissue were more distinct, and inflammatory infiltration of the dermis adjacent to the wound was insignificant. Epithelial regeneration was found in all of the wounds. The regenerating epithelium was rather extended, with clear signs of multi-layer differentiation, and epithelium grew in some areas without complete cleaning of the bottom with epithelium growing under the pellicle.

Day 15

a) Untreated Control group. Only 20% of the animals demonstrated significant cleaning of wounds and distinct but uncompleted epithelialization: the central part of the wound was devoid of epithelium, and the bottom had mature connective tissue with a tendency towards cicatrisation. 80% of the animals had wounds with incomplete cleaning of the surface and distinct inflammatory infiltration of the mature connective tissue, with uneven cicatrisation on various levels of the regenerating connective tissue. Granulation tissue was observed in the areas of distinct inflammatory infiltration, with leucocytes topographically corresponding to the zones which did not show signs of complete cleaning. Epithelial regeneration was weak, with wedge-shaped extension of the epithelial layer out of the wound edges. Pseudoepitheliomatous hyperplasia was seen in the epidermis of the skin areas adjacent to the wound. This is illustrated in FIG. 1A.

b) Placebo. 60% of the animals showed complete cleaning of the wound surface and wound epithelialization. However, multi-layer flat regenerating tissue was infiltrated with leucocytes, with oedema and clear signs of dystrophy. These areas correspond topographically to the foci of pronounced oedema, hyperaemia, inflammatory infiltration of the mature connective tissue at the bottom of the wound. Sclerosis and granulomas of foreign substances were found in the fatty tissue. The epidermis adjacent to the wound showed pseudoepitheliomatous hyperplasia. 40% of the animals demonstrated weak cleaning, with only the edges of the wound being epithelialized, which corresponds with intense inflammatory infiltration of the connective tissue regenerant.

c) Compound 2

60% of the animals showed complete epithelialization of the wound, with a considerable extent of differentiation of the regenerating epithelium forming a multiple flat layer over the entire wound area. Under the multi-layered flat epithelium, there was mature connective tissue with normal collagen fibre architecture, and no signs of disorganisation and cicatrisation. Cellular infiltration of the regenerating connective tissue was minimal. Clear boundaries could be observed between the bottom of the healed wound, sub-epidermal cellular tissue and adjacent areas of dermis. 40% of the animals showed incomplete epithelialization on a considerable area of the bottom of the wound. In this case, there were patchy remains of pellicle on the surface of non-epithelialized areas. The epithelial regenerant sometimes grew under the pellicle and detached it from the wound. In such areas, there was focal lymphocyte, leucocyte and macrophage infiltration. This is illustrated in FIG. 1B.

### EXAMPLE 16

### Morphological indicators of wound healing

A comparative assessment of the healing of a full-thickness wound was performed on the basis of a set of morphological indicators characterising the regeneration process:

(a) timeframe and features of wound cleaning, and secondary infection of the aseptic wound;

(b) timeframe, extent, direction and the level of differentiation of epithelial regeneration at the bottom of the wound;

(c) time of development, topography, and spreading of connective tissue regenerant;

(d) presence and degree of inflammation in the regenerant;

(e) presence or absence of synchronisation of the regeneration processes in the epithelium-connective tissue system;

(f) presence or absence of morphological signs of pathological regeneration of epithelium in connective tissue (acanthosis, pseudoepitheliomatous hyperplasia, horny cyst, cicatrisation); and

(g) correlation between these processes and the type of wound healing (primary or secondary intention).

Skin wounds were created and treated in all the animals in each group, as described in Example 14. All the indicators listed above varied within the group. However, these differences highlighted certain tendencies inherent in the five groups of wounds. Thus the untreated control group showed the following:

1. Delayed or incomplete cleaning of the massive pellicle (necrotic masses, fibrin, leucocytes) from the bottom of the wound, which was observed with some animals even at the fifteenth day of the experiment;

2. Epithelial regeneration was weak and stabilised in the boundary areas of the wound, and epithelial regeneration did not demonstrate a tendency to differentiation of flat cells;

3. Considerable growth of the granulation tissues on the bottom of the wound, with uneven maturation and consistent inflammatory infiltration;

4. Lack of simultaneous proliferation in epithelium and connective tissue, which, in conjunction with current inflammation, determines the tendency to healing by secondary intention.

Morphological data obtained in the placebo groups (lanolin ointment base) were similar to those in the untreated groups on the fifth and tenth days. However, a positive dynamic was observed on the fifteenth day: the wounds recleaned and epithelialized in a considerable area, and regenerating connective tissue had matured. However, the tendency towards inflammation remained, and the inflammation spread to the epithelium, which reduced the possibility of epithelial proliferation and differentiation and encouraged wound healing by secondary intention. This is illustrated in FIG. 1A.

Considerable differences were observed when studying the healing of wounds with the test compound. These differences supplement and confirm the results on the positive healing effect of Compound 2 on full-thickness skin wounds. As early as the fifth day, the compound induced focal cleaning of the wound surface from the pellicle and the formation of epithelium regenerant with signs of differentiation of flat cells at the wound edges. On the tenth day, simultaneous regeneration of epithelium and connective tissue and a tendency towards even maturation of regenerant was clearly observed. Even when the pellicle partially remained on the wound surface, epithelial regenerant grew under it. By the fifteenth day, most animals demonstrated the completion of the healing process by primary intention, with differentiation of flat cells, and no signs of pathological regeneration or cicatrisation of connective tissue. This is illustrated in FIG. 1B.

Spasatel resulted in less favourable healing than Solcoseryl, and was markedly less effective than compound 2. The cleaning of the surface was slow and incomplete, even on the fifteenth day, and correlated with constant, extensive inflammatory infiltration of the connective tissue regenerant. In a number of cases, during the inflammation, the simultaneity of regeneration in the epithelium-connective tissue system was disrupted, the surface was incompletely epithelialized, and connective tissue was cicatrised in some foci, creating prerequisites for healing by secondary intention. This is illustrated in FIG. 1C.

Solcoseryl was comparable to Compound 2 in proliferation potential. However, with Solcoseryl the pellicle was detached and the wound was cleaned at a later stage, which created conditions which promoted inflammation in the regenerating connective tissue. This correlated with the incomplete epithelialization of the wound, and the possibility of leucocyte degradation of the regenerating epithelium, which creates prerequisites for healing by secondary intention. This is illustrated in FIG. 1D.

### EXAMPLE 17

### Treatment of wounds in human patients

The effect of Compound 2 on the healing of different types of wounds was tested in human patients. A powder, solution or ointment of the substance containing the active agent in a proportion of 5% to 95% was applied to the wound. In all cases the wounds healed rapidly without infection or scarring.

1. Patient A. 38 Years Old (Incisional Wound 5 cm)

The wound surface was treated with 10% Compound 2 in a lanolin ointment base 12 hours after the wound was made. 1 g of the ointment was applied under a bandage. The procedure was repeated daily for 5 days. The wound closed over, and at 17 days there was no scar, and the skin had fully recovered.

2. Patient B. 78 Years Old (Bed Sores on the Thigh 1.0×2.0 cm)

0.5 g of a finely dispersed powder of Compound 2 was applied to the surface of the wound under a bandage. The procedure was repeated daily for 5 days. The wound closed over, and at 16 days there was no scar, and the skin had fully recovered.

3. Patent C. 15 Years Old (Second Degree Thermal Burn on the Left Hand, 2.0×1.5 cm)

A sterile napkin wetted with a 10% sterile aqueous solution of Compound 2 was applied to the wound 10 minutes after the burn. The napkin was rewetted as it dried. The procedure was repeated within 24 hours. In one hour, the pain at the place of burn had stopped. On the next day it was observed that hyperaemia had been considerably reduced at the site of the burn. Examination at 16 days demonstrated complete recovery of the skin, without scars.

4. Patient D. 9 Years Old (Abrasion of the Left Leg, 3.0×0.5 cm))

0.5 g of a finely dispersed powder of Compound 2 mixed with an equal amount of talc was applied to the surface of the wound under a bandage. The procedure was repeated after 5 days. The wound closed over, and at 12 days the skin had healed completely.

5. Patient E. 68 Years Old (Erosion of the Uterine Cervix, 1.5×1.2 cm)

The surface of the wound was dried, and 10% Compound 2 ointment, prepared as for patient A, was applied to the wound surface on a tampon. The procedure was repeated daily for two weeks. A colposcopy at 18 days showed that the epithelium of the mucous membrane of the uterine cervix had fully recovered.

### EXAMPLE 18

### Effect of Compound 2 on the healing of mouth ulcers

A model of mouth ulcers using ulcers caused by cryogenic injury of the inner surface of the cheek was used for this purpose. Tests were performed on 110 male rats weighing 180-200 g, with 10 animals in each group. Ulcers were created in experimental animals by pressing a metal rod of 2.5 mm in diameter cooled in liquid nitrogen to the mucosa for 10 seconds under ether anaesthesia. Animals were kept in separate cages, with normal food and water ad libitum. Treatment was administered by spraying of the mucous membrane of the mouth, 24 hours after wounding and then daily until healing was complete. Compound 2 was administered to the animals at a dose of 50 mg/kg (2 ml of aqueous solution). The following groups of animals were used:

Group 1: intact animals;

Group 2: cryogenic injury (wound) on day 1;

Group 3: wound on the 3rd day;

Group 4: wound+treatment on day 3;

Group 5: wound on day 7;

Group 6: wound+treatment on day 7;

Group 7: wound on day 14;

Group 8: wound+treatment on day 14.

Cryogenic damage to the oral mucosa results in a wound characterized by a necrotic surface, inflammation with intensive leukocyte infiltration, pronounced blood circulation disorders, fibrinoid necrosis of the connective tissue of the mucosa and expansion of the inflammation into the muscle, and reactive skin inflammation.

The progress of wound healing was examined morphologically on days 1, 3, 7 and 14. On the day 3 the lesions were progressively worse, resulting in the growth of necrotic changes with the formation of a wide necrotic zone at the bottom of the wound, and growth and expansion of acute inflammation involving the muscle, skin and mucosa beyond the wound area. On day 7, the wounds in half of the animals were partially cleared of the necrotic mass, accompanied by the development of granulation tissue at the bottom of the wound and in the adjacent mucosa. There were initial signs of regeneration of multilayer flat epithelial cells at the edges of the wound, and decreased signs of inflammation. On days 7 and 14 in particular, there was an increased tendency towards wound surface cleaning and connective tissue development and maturation. However, focal inflammatory activity with focal purulent fusion of the maturing granulation tissue remained in the surface areas of the wound, particularly under the necrotic mass. Epithelialization of the wound did not exceed the boundaries of the initial stage, taking place at the edges of the wound. There was no sign of a tendency towards complete epithelialization. These results are illustrated in FIG. 2A.

A comparison of the experimental group treated with Compound 2 and the control group demonstrated that on day 3 the wound was free of necrotic mass and that there was an increase in granulation tissue, correlating with subsiding inflammation, as well as epithelial regeneration at the edges of the wound. Wound healing processes in the experimental group were even more distinct on day 7.60% of the animals showed complete cleaning of the wound surface, which was accompanied by reduced inflammation, maturation of granulation tissue, reduction of size of the wound defect, as well as more pronounced signs of epithelial regeneration. These results are illustrated in FIG. 2B.

On day 14, these tendencies were more pronounced: there were no wounds with incomplete cleaning of necrosis from the bottom, and there were no signs of active inflammation on the bottom and adjacent tissue. The size of the wound surface was significantly reduced; maturation of the granulation tissue on the bottom and in the adjacent mucosa correlated with a tendency towards complete epithelialization of the wound surface in all animals, without any scars or deformity. These results are illustrated in FIG. 2C.

Therefore comparative morphological analysis indicated that Compound 2 induced a more pronounced, earlier and more complete healing of a cryogenic injury of the rat oral mucosa compared to untreated controls.

### EXAMPLE 19

### Effect of Compounds 1-3 on repair processes in the rat gastric mucosa

Experiments in rats using various models of stomach damage indicated that compounds 1, 2 and 3 have a prophylactic and therapeutic effect, as summarized in Table 18. The models used gastric ulcers induced by

a) Electrostimulation of immobilised rats for 3 hours;

b) Immobilization and hypothermia (induced by water cooling);

c) Ulcer created by local wounding with a cryogenic probe using a method similar to that described above for mouth ulcers.

The compounds were administered intraperitoneally at a dose of 20 mg/kg; in the cryogenic ulcer model compound 2 was administered either intraperitoneally (10 mg/kg) or orally (20 mg/kg twice daily, ie. a total of 40 mg/kg/day) and was compared to orally-administered gastrocepin (1 mg/kg twice daily, ie. a total of 2 mg/kg/day).

However, in contrast to Compound 1, Compound 2 significantly reduced the frequency, size and severity of the injury of the mucosa. Compound 3 did not affect gastric secretory function in dogs or rats in either chronic or acute tests.

All three compounds accelerated repair processes in the gastric mucosa. As shown in Table 18, after 3 days rats treated with these compounds had 1.2-2 fold less erosive injury than untreated animals.

The cryogenic ulcer model demonstrates that by day 14 Compound 2 at a dose of 10 mg/kg reduced the size of lesions in the gastric mucosa by 53% when administered intraperitoneally, and by 51% when a daily dose of 40 mg/kg was administered orally. The effect of Compound 2 was similar to that of the conventional ant-ulcer agent Gastrozepin (pirenzepine; Thomae).

Both prophylactic and therapeutic administration of Compound 2 to animals reversed the changes in content of the enzymes cytochrome oxidase and succinate dehydrogenase, whose level is an index of the status of the oxidative phosphorylation and functional activity of the gastric mucosa.

TABLE 18
PROTECTIVE AND HEALING EFFECT OF COMPOUND
1, 2 AND 3 ON LESIONS OF THE GASTRIC MUCOSA IN RATS

| | | | | |
|---|---|---|---|---|
| | | % | | % |
| | Average | reduction | | reduction |
| | number of | of | | of |
| | lesions per | degree of | Area of | ulcerated |
| Types of Lesion | animal | ulceration | lesion | area |

Protective effective 30 minutes before the test

| | | | | |
|---|---|---|---|---|
| Electric stimulation of | | | | |
| immobilised animals | | | | |
| Control | 7.0 ± 0.7 | 0 | 15.2 ± 5.7 | 0 |
| Compound 1 | 7.0 ± 0.7 | 0 | 15.0 ± 4.7 | 0 |
| Compound 2 | 2.4 ± 0.8 | 66 | 2.8 ± 2.1 | 91 |
| Compound 3 | 3.5 ± 0.4 | 50 | 7.1 ± 4.1 | 54 |
| Immobilisation of | | | | |
| animals under | | | | |
| hypothermia | | | | |
| Control | 11.5 ± 1.5 | 0 | 10.4 ± 2.4 | 0 |
| Compound 1 | 10.2 ± 1.2 | 0 | 10.1 ± 1.4 | 0 |
| Compound 2 | 4.1 ± 0.9 | 64 | 4.1 ± 0.8 | 62 |
| Compound 3 | 6.8 ± 1.4 | 41 | 7.2 ± 2.0 | 41 |

Healing effect

| | | |
|---|---|---|
| Electric stimulation of | | |
| immobilised animals | | |
| (healing effect over | | |
| the course of 3 days) | | |
| Control | 3.4 ± 0.2 | 0 |
| Compound 1 | 2.8 ± 0.7 | 18 |
| Compound 2 | 1.7 ± 0.2 | 50 |
| Compound 3 | 2.5 ± 0.4 | 26.5 |
| Cryogenic ulcer | | |
| (healing effect over the | | |
| course of 14 days) | | |
| Control | 18.6 ± 2.2 | |
| Compound 1 | 8.8 ± 1.9 | 53 |
| Compound 2 | 9.4 ± 1.0 | 62 |
| (twice daily, oral) | | |
| Compound 3 | 7.7 ± 2.5 | 59 |
| (twice daily, oral) | | |

### EXAMPLE 20

### Clinical studies of the effect of Compound 2 on gastric and duodenal ulcer.

A clinical trial of the effect of Compound 2 on gastric and duodenal ulcer was carried out in several gastroenterological clinics in Russia. A total of 167 patients, 30 to 75 years old, was studied; of these 93 had duodenal ulcers and 74 had gastric ulcers. The results were monitored by gastroscopy at weekly intervals. Each patient continued to receive the treatment which s/he had been given prior to commencement of the trial. Compound 2 was administered in addition to the previous treatment. Compound 2 was given orally as three 0.2 g tablets per day over a period of three weeks, ie a total dose of 12 g for the treatment course, Compound 2 healed 80% of all the duodenal ulcers treated, and 54% of all the gastric ulcers treated. Compound 2 was also effective in cases which had been found to be resistant to conventional therapy. The effect of Compound 2 was compared with those of a placebo, or cimetidine. Even better results were obtained when Compound 2 was administered together with conventional therapy. No side effects were observed. Following termination of treatment with Compound 2 no ulceration was observed, ie there was no sign of the abstinence syndrome which is typically seen with H₂-blockers and hormones. Compound 2 was an effective therapeutic agent with a pronounced reparative effect on erosive and ulcerating injuries of the gastroduodenal area.

### EXAMPLE 21

### Effect of Compound 2 and Compound 6 in a model of ulcerative colitis

A model of cryogenic damage of the colon was used in this experiment to simulate ulcerative colitis. Cryogenic injuries of the sigmoid colon of rats were induced by pressing a metal rod of 2.5 cm in diameter cooled in liquid nitrogen to the serous membrane of the colon for 10 seconds under ether anaesthesia.

Tests were performed on 67 male rats weighing 180-200 g, with 8 animals in each group. Animals were kept in separate cages, with normal food and water ad libitum. The test compounds were administered orally on the second day after the operation, and then daily until healing was complete. Compound 2 or Compound 6 was administered to test animals at a dose of 20-50 mg/kg. The following agents were used for comparison:

50 mg/kg of Potassium orotate+50 mg/kg of Riboxin (inosine);

180 mg/kg of Methyl uracil;

2 g/kg of Sulfasalazine (Pharmacia)

The following groups of animals were used:

Group 1: intact animals days 1, 3 and 7;

Group 2: ulcer untreated control days 1, 3 and 7;

Group 3: ulcer+Compound 220 mg/kg orally days 3 and 7;

Group 4: ulcer+Compound 250 mg/kg days 3 and 7;

Group 5: ulcer+Compound 620 mg/kg days 3 and 7;

Group 6: ulcer+Potassium orotate 50 mg/kg+Riboxin 50 mg/kg orally days 3 and 7;

Group 7: ulcer+(Methyl uracil 180 mg/kg orally) days 3 and 7;

Group 8: ulcer+(Sulfasalazine 2 g/kg orally) days 3 and 7

The nature of the healing of the ulcer injury was examined morphologically on days 1, 3 and 7. The morphological analysis showed that in the control group of animals damage of the colon wall was produced, with destruction of the mucosal and muscular layers and development of a necrotic process with a total lesion of the mucosa; formation of a leukocyte necrotic scab; significant lesion of the muscular membrane; and acute serosal, fibrocytic, and leukocyte inflammation in the damaged area, including the peritoneum. The inflammatory process extends beyond the injured area into the adjacent areas, and beyond the peritoneum into fatty connective tissue.

In the control animals the healing process has the following dynamics:

On day 1, there was comprehensive injury, with large necrotic surfaces and no signs of rejecting the necrotic mass, and a pronounced diffuse infiltration of leucocytes into the sub-necrotic zone. The wall of the colon was totally damaged, with an acute oedema and leucocyte infiltration. Acute inflammation extended into the peritoneum, adjacent cellular tissue and colonic areas. The edges of the necrotic zone were represented by the mucosa of the adjacent areas of the colon.

On day 3, there was a tendency to reject the scab, which was expressed differently in various animals; however, there was no instance of complete cleaning from necrosis. There were no signs of epithelial regeneration in the majority of the animals. The damaged areas showed pronounced inflammation, together with increasing granulation and initial signs of maturation.

On day 7, there was a significant cleaning of necrosis from the bottom of the injured area. The submucosa and muscular layers were replaced by granulation with different extents of maturity in conjunction with inflammatory infiltration, which remained in both the injured and adjacent parts of the colon. The size of the wound was reduced, and there was incomplete regeneration of the mucosa without differentiation. In some cases there were focal remnants of the scab. These results are illustrated in FIG. 4A.

In accordance with these results, we have identified the following criteria for comparative evaluation of the healing processes in wound areas treated with different agents:

1. Scope of necrotic alterations, and scab cleaning dynamics of the wound;

2. Degree and extent to which inflammation is found in the wound area;

3. Dynamics, quality and synchrony of repair processes—regeneration of the epithelium and connective tissue and quantitative completeness of regeneration.

Animals treated with the agents used for comparison (Methyl uracil, Potassium orotate+riboxin, Sulphasalazine) did not significantly differ from the control groups on day 3; on day 7 they were characterised by a slower cleaning of necrosis from the bottom of the wound, weak epithelial regeneration, lack of synchrony of epithelial and connective tissue regeneration, and persisting inflammation compared to controls. On day 7 18% of animals demonstrated complete healing.

A comparison between the experimental groups treated with Compound 2 or Compound 6 and controls clearly demonstrated a number of major differences. Beginning on day 3, there was an accelerated rejection of the scab from the surface of the wound, and reduction of the intensity of inflammation in the wound area and beyond. On day 7, there was an acceleration in the granulation growth and maturation in the area of damage. When Compound 2 at a dose of 20 mg/kg was used, there was a tendency on day 7 towards complete regeneration of the epithelium and mucosa and reduction of the wound area due to synchronous regeneration, which was complete in 100% of cases, as illustrated in FIG. 4B, whereas a dose of 50 mg/kg resulted in a reduction of the wound area in only 67% of animals, as shown in FIG. 4C.

All the signs listed above were observed in all animals in the test groups; however, their extent and timing varied between different groups. The optimal indicators of healing were observed when Compound 2 was used at doses of 20 mg/kg and 50 mg/kg; they were less pronounced with Compound 6.

### EXAMPLE 22

### Effect of Compound 2 in a model of myocardial infarction

The effect of Compound 2 on myocardial tissue in an experimental model of myocardial infarction was examined. In this model system neurogenic dystrophy of the myocardium was induced in rats or rabbits by electrical stimulation of the reflexogenic zone of the aortic arch by implanted electrodes for a period of three hours. This treatment induces functional disturbances of the activity of myocardial muscle, which are characterised by changes in the amplitude of the QRS waves and the position of the ST segment as demonstrated by 12-lead electrocardiography. These changes are accompanied by changes in biochemical indicators such as creatine phosphate, cAMP, and calcium ion concentrations, and changes in the activity of redox enzymes such as succinate dehydrogenase, lactate dehydrogenase, and glucose-6-phosphate dehydrogenase. At a later stage organic lesions of the myocardium such as necrotic and fibrosclerotic foci can be observed.

The effect of Compound 2 given intraperitoneally or orally on these indicators was examined in rats and rabbits following electrostimulation as described above. The effects on levels of creatine phosphate in rat myocardial are shown in Table 19, and the effects on adenosine triphosphatase (ATPase), succinate dehydrogenase and glucose-6-phosphate dehydrogenase rabbit myocardium are shown in Table 20. The effects on cAMP and Ca⁺⁺ levels in rabbit myocardial tissue are shown in FIG. 5, and effects on creatine kinase in rabbit myocardium and blood and the levels of creatine phosphate in myocardium are shown in FIG. 6.

TABLE 19
Level of creatine phosphate (μM/g) in rat myocardium
following intraperitoneal administration of compound 2.

| Conditions of | Intact | | Compound 2 | |
|---|---|---|---|---|
| Experiments | animals | Control | 10 mg/kg | 20 mg/kg |
| Before | 1.22 ± 0.06 | 0.50 ± 0.07 | 0.92 ± 0.08 | 1.04 ± 0.15 |
| stimulation | | | | |
| 3 days after | 1.07 ± 0.05 | 0.54 ± 0.05 | 0.95 ± 0.06 | 1.07 ± 0.04 |
| stimulation | | | | |

| | | | Oral Compound 2 |
|---|---|---|---|
| | | | 10 mg/kg |
| Before | 1.10 ± 0.04 | 0.47 ± 0.04 | 0.80 ± 0.07 |
| stimulation | | | |
| 3 days after | 1.19 ± 0.08 | 0.76 ± 0.08 | 1.09 ± 0.1 |
| stimulation | | | |

TABLE 20
Effect of Compound 2 on activity of enzymes in rabbit myocardium

| | | 48 hours after electrostimulation | | |
|---|---|---|---|---|
| Experimental | Intact | Without | Compound 2 | Ethimizol |
| marker | animals | treatment | 10 mg/kg | 5 mg/kg |
| ATPase | 0.57 ± 0.13 | 0.28 ± 0.12 | 0.47 ± 0.13 | 0.43 ± 0.11 |
| (μM/mg/min) | | | | |
| | P < 0.001 | — | P < 0.01 | P > 0.05 |
| Succinate | 46.1 ± 5.8 | 31.8 ± 6.2 | 44.2 ± 6.7 | 39.7 ± 7.4 |
| dehydrogenase | | | | |
| (optical density | | | | |
| units) | | | | |
| P | P < 0.001 | — | P = 0.01 | P > 0.05 |
| Glucose-6- | 5.6 ± 0.6 | 7.1 ± 0.8 | 6.2 ± 0.7 | 5.8 ± 0.7 |
| phosphate | | | | |
| dehydrogenase | | | | |
| (optical density | | | | |
| units) | | | | |
| | P < 0.01 | — | P > 0.05 | P = 0.05 |

Note:
P is estimated in comparison with the group of rabbits which were not treated after electrostimulation.

These results show that Compound 2 is effective both orally and intraperitoneally in minimising myocardial damage in this model. Control animals, which did not receive Compound 2 before or after electrostimulation, showed a reduction in biochemical indicators of myocardial damage, and also showed necrosis, proliferation of the myocardial stroma, and sclerotic centres, ie scar formation. The effect of Compound 2 was comparable to that of Riboxine or levodopa.

### EXAMPLE 23

### Effect of Compound 2 in patients with myocardial infarction

A clinical trial of Compound 2 was carried out on 100 patients aged from 25 to 75 years during the subacute period of myocardial infarction. Each patient continued to receive the treatment which s/he had been given prior to commencement of the trial. Compound 2 was administered in addition to the previous treatment. A control group received conventional treatment, ie. nitrate, anticoagulants and/or hypotensive agents as appropriate. The results of treatment of these two groups were compared with groups of patients receiving riboxine and placebo. Compound 2 was administered over a period of three weeks from the 7th to 28th days following infarction, and was administered orally at a dose of three 0.2 g tablets per day.

The effects of treatment were monitored by 12-lead electrocardiography, using the QRS Scoring System. Haemodynamic parameters and the contractile function of the myocardium were monitored using a Toshiba ultrasound camera model SSH-60A, using conventional methods.

The effect of Compound 2 was also studied in 25 patients aged 43 to 66 years suffering from cardiac insufficiency, in comparison with riboxine or a placebo.

In the patients suffering from myocardial infarction, recovery of the contractile function of the myocardium was more rapid in patients receiving Compound 2 than in patients receiving placebo or riboxine. In patients suffering from cardiac insufficiency, Compound 2 prevented the progression of the condition, and prevented dilatation of the left ventricle. No side effects were observed in either group.

### EXAMPLE 24

### Effect of Compound 2 in patients suffering from myocardial infarction

The effect of Compound 2 therapy in patients suffering from the subacute phase of myocardial infarction (MI) was assessed. The patients were divided into two groups. There were no substantial differences between the groups in terms of age and basic features of necrotic foci. Each patient continued to receive the treatment which s/he had been given prior to commencement of the trial. Compound 2 was administered in addition to the previous treatment. The first group included 63 patients who received conventional therapy, i.e. nitrates, anticoagulants and/or hypotensive agents as appropriate, and the second group comprised 44 patients who were treated with Compound 2 at a dose of 600 mg per day from the 7th to the 28th day after diagnosis of myocardial infarction, in addition to receiving conventional treatment. The control group included 14 healthy men aged from 26 to 42. The average age of patients in the first group was 50.3 years, and in the second group, 49.5 years.

Repeated myocardial infarctions were diagnosed for 20% of the first group and 25% of the second group. The rate of localization of necrotic foci in the anteroseptal and anterolateral regions of the left ventricle of the heart was 0.47 in the first group and 0.38 in the second group. The inferior and inferiolateral localization of the necrotic foci was recorded at a rate of 0.53 for patients in the first group and at a rate of 0.62 for patients in the second group.

Clinical evidence from daily examinations of both groups and the results of electrocardiagraphic examination in 12 abductions were assessed over the entire observation period. A number of analyses were made on the 7th and 28th day of progress of MI, including clinical blood count, plasma bilirubin, sugar, creatinine, cholesterol, β3-lipoproteins, potassium and sodium, and urine analysis; the activity of the prothrombin complex and the time of re-calcification were also determined. Central haemodynamics were examined using the method of body integral rheography, whereby the stroke volume and cardiac indices were calculated. Indices of left ventricular contractility—terminal diastolic volume, ejection fraction, and the extent of systolic contraction of anteroposterior cavity dimensions (% ΔS)— were examined using the echography method using an Aloka SSD-119 apparatus (Japan). Central and intracardiac haemodynamics was investigated on the 1st, 7th; 14th, 21st and 28th days of the disease. Physical examination data showed no substantial differences between the groups of patients during the observation period.

Clinical tests of Compound 2 showed that treatment of myocardial infarct patients over the course of three weeks (from the 7th to the 28th day of the disease), with a dose of 600 mg per day, did not produce any clinical side effects. Compound 2 therapy resulted in an increase in unconjugated and hence total bilirubin in serum, which does not, however, exceed the upper limit of standard values. Combining Compound 2 therapy with conventional therapy of MI patients increased the rate and extent of regeneration of left ventricular myocardial contractility. Our results indicate that Compound 2 is useful for treating MI patients in the subacute phase.

### EXAMPLE 25

### Comparison of clinical efficacy of Compound 2 and Riboxin in the subacute phase of myocardial infarction

A total of 94 patients were examined; of these:

Compound 2 was given to 32 patients;

Placebo was given to 14 patients;

Riboxin (inosine) was given to 18 patients;

Conventional therapy as for example 24 was given to 30 patients. Each patient continued to receive the treatment which s/he had been given prior to commencement of the trial. Compound 2 was administered in addition to the previous treatment.

All the patients underwent standard laboratory tests prior to commencement of Compound 2 therapy, 14 days after the beginning of the course and upon completion of the course. The tests included clinical blood analysis; general urine analysis; evaluation of creatinine, bilirubin, transaminase activity, blood sugar, cholesterol and β-lipoproteins, malonic dialdehyde, Schiff bases, zinc, iron, copper, plasma potassium and sodium, prothrombin and fibrinogen; and an ECG examination in dynamics.

All the patients received conventional therapy, i.e. nitrates, anticoagulants and/or hypotensive agents as appropriate.

Combination therapy included Compound 2 (1st group—32 patients) or Riboxin (2nd group—18 patients). The agents were administered at a dose of 200 mg three times per day.

In addition to an improvement of subjective data, Compound 2 produced a positive effect on the myocardial contractile function, since there was a clear increase in the ejection fraction from 54.4±1.5% (an the absence of the agent) to 60.0±0.5% (approx. 6%), in contrast to the group of patients who were treated with Riboxin (56.8±1.3%).

The patients treated with Compound 2 showed a tendency toward the normalization of cholesterol levels at 96.5% and a decrease in the level of prothrombin to 91.3%, whereas the figures for patients who were treated with Riboxin were 85.5% and 88.7%, respectively.

The most pronounced therapeutic effect was obtained for patients with repeated myocardial infarct, in whom cicatrisation took place one or two days earlier (27 days on the average) in 90% of cases, than patients who were treated with Riboxin (12% of cases).

A study of the electrolyte composition of blood, in particular microelements such as zinc and copper, produced fairly interesting results. As is known, myocardial infarct causes a decrease in the level of zinc. Patients who took Compound 2 showed a substantial increase in the zinc level compared to the Riboxin group, in whom zinc remained below normal levels throughout the entire course of therapy. Furthermore, it was found that during the treatment with Compound 2 the level of copper increased proportionally to the increase in the content of superoxide dismutase, an antioxidant plasma enzyme, which reflects normalization of homeostasis. The patients who took riboxin showed no such relationship.

Intensification of lipid peroxide (LPO) oxidation processes accompanied by lower activity of antioxidant systems plays a prominent role in the pathogenesis of ischaemic cardiac disease. All the patients demonstrated an initially high level of LPO indicators (malonic dialdehyde and Schiff bases). Before treatment, all the patients had above normal malonic dialdehyde levels, i.e. 1.3 nmole/ml on average, with the normal level being 1.21 nmole/ml. During treatment with Compound 2 there was a clear decrease in malonic dialdehyde to 1 nmole/ml on the 21st day. A decrease in the malonic dialdehyde level during treatment with riboxin is consistent with progression of the myocardial infarct. The level of Schiff bases before the treatment was 75 conventional units, ie above normal (60 conventional units). After Compound 2 therapy, the level of Schiff bases fell sharply to 40 conventional units, compared to 62.6 conventional units after completion of Riboxin therapy. Before treatment, superoxide dismutase activity was low in both groups, being on average 18-20 conventional units. After Compound 2 therapy, the superoxide dismutase levels exceeded the norm (23 conventional units), reaching 25 conventional units; after Riboxin therapy, however, this parameter not only did not increase, but actually fell slightly, to 21.5 conventional units.

All the patients who were treated with Compound 2 felt well, and did not develop any allergic reactions or side effects.

### EXAMPLE 26

### Effect of Compound 2 on carbon tetrachloride-induced liver damage

The prophylactic and therapeutic effects of Compound 2 on liver damage were examined in a carbon tetrachloride intoxication model. Liver dysfunction was induced by the intravenous injection of a liquid paraffin-based 50% carbon tetrachloride solution at a dose of 0.8 ml per 100 g of body mass daily for four days. Inbred male white rats weighing 200-220 g were treated with Compound 2 at a dose of 20 mg/kg given orally by intragastric tube on the eighth day after injection of carbon tetrachloride, and daily for four days thereafter. Control animals received carbon tetrachloride only. Animals were sacrificed 7 days after the last injection of carbon tetrachloride, when the morphological changes are most pronounced, and samples of liver were taken for histology.

In the control animals necrosis of the liver parenchyma and destruction of the characteristic architecture were observed; in addition, fatty dystrophy and diffuse inflammatory infiltration of the portal stroma were seen. Mitotic figures were not observed. In animals treated with Compound 2, the degree of necrosis was less, and the necrotic zones were localized rather than confluent; albuminous dystrophy and central inflammatory infiltration of the portal stroma predominated. Giant mononuclear, binuclear and polynuclear hepatocytes were found in all sections, and mitotic figures were frequent. Thus compound 2 has a protective effect on the parenchyma of the liver and increases liver regeneration following carbon tetrachloride intoxication, suggesting that the compounds of the invention may be useful in the treatment of chronic active hepatitis. These results are summarised in Table 21.

TABLE 21
Comparison of the main morphological indicators of control
and experimental animal groups following intoxication with CCI₄

| | |
|---|---|
| Indicators | Groups of animals |

| (arbitrary) | Control | Compound 2 | P |
|---|---|---|---|
| Necrosis of | 8.2 ± 0.24 | 2.7 ± 0.3 | <0.001 |
| hepatocytes | | | |
| Elimination of | 1.8 ± 0.2 | 4.9 ± 0.4 | <0.001 |
| glycogen from | | | |
| damaged zones | | | |
| Average mitotic | 0.0012 ± 0.001 | 0.021 ± 0.001 | <0.001 |
| index | | | |

### EXAMPLE 27

### Reparative effect of Compound 2, Compound 4, Compound 5, and Compound 6 after partial hepatectomy

Partial hepatectomy was performed using the conventional method (Higgins and Anderson, 1931) by removing the left lateral and central lobes of the liver from 80 male white rats, weighing 180-200 g.

Compound 2, Compound 4, Compound 5 or Compound 6 was administered to the experimental groups intraperitoneally in equimolar doses of 0.1 mM/kg and 0.2 mM/kg. These optimal doses were determined in preliminary experiments. The control group received intraperitoneal injections of saline solution. The compounds were administered on the day after the operation and then one injection was given daily over the course of 7 days, since maximum growth of the liver mass in rats is observed in the first 7 days after partial hepatectomy (Solopaev, 1980).

The effectiveness of the compounds was compared to that of a combination of non-steroid anabolic regeneration stimulators which are used in the treatment of liver pathology, namely Riboxin and potassium orotate at a dose of 0.2 mM/kg each (Rychnev and Frolov, 1984).

For quantitative evaluation of the regeneration process in the liver the coefficient of regeneration completion as determined by the formula below was used:

$$K = \frac{{P}_{1} - {P}_{2}}{{P}_{3}} \cdot 100 \% ,$$

where

P₁=Liver mass 7 days after partial hepatectomy

P₂=Remaining liver mass after partial hepatectomy

P₃=Mass of liver removed

The initial liver mass which is necessary in order to calculate the mass of the organ remaining after the operation was calculated based on the total body mass of the animal, because liver mass in rats is directly proportional to the body mass.

The regression equation is as follows:
Y=0.036x+2.37,

where x=mass of the animal, and y=liver mass The coefficient of linear correlation for evaluation of the size being investigated is 0.654 (Gaivoronskaya et al, 2000).

Stimulation of the regeneration of tissue after hepatectomy in response to different medicinal preparations is associated with an increase in the synthesis of nucleic acids, as indicated by the increase in the content of RNA and DNA in the regenerated tissue. Quantitative analysis of DNA and RNA was performed by centrifugation. Comparative analysis of the content of DNA in healed animals was found to be the most informative. The enzymic component of anti-oxidant protection was also assessed on the basis of the activity of catalase and superoxidedismutase (SOD).

RNA and DNA Content

5 ml of 0.3 MM/kg of HCIO₄ solution were added to the sample (100 mg). In order to ensure complete sedimentation of the acid-insoluble fraction, the beakers were put in ice for 15 minutes. Then they were centrifuged for 10 minutes at 5000 revolutions per minute. The sediment was washed twice with 0.2 M HCIO₄. After the final centrifugation, the walls of the beaker were carefully dried with gauze and filter paper. The sediment was ground down with a glass pestle, suspended in 1 ml of water and then 1 ml of 0.6 M KON solution was added at room temperature. Hydrolysis was performed over the course of 1 hour at 37° C., then the beakers were put in ice to stop the hydrolysis process. 4 ml of the 0.6 M solution were added to each beaker and the beakers were left in ice for another 15 minutes, then centrifuged for 15 minutes at 5000 revolutions per minute. The supernatant was used to determine the RNA by UV absorption at 260 nm and 290 nm compared to the control sample containing 0.4M of HCIO₄). The amount of RNA in μg per 1 ml of the supernatant was calculated using the following formula:

$$C = \frac{{D}_{270} - {D}_{290}}{0.19} \times 10.5$$

where C=is the RNA concentration (μg/ml)

DNA analysis was carried out on the sediment remaining after the alkaline hydrolysis used to determine RNA. 0.5 M of HCIO₄ (5 ml per sample) was poured into the dried beaker containing the sediment. Hydrolysis was performed in a boiling water bath for 20 minutes. The amount of DNA in μg per 1 ml of the hydrolysate was calculated using the following formula:

$$C = \frac{{D}_{270} - {D}_{290}}{0.19} \times 10.1$$

where C=is the DNA concentration (μg/ml).
Catalase Activity

Catalase activity was determined using the permanganate method. The optimal amount of the substrate (3 ml of 1% H₂O₂) and 1 ml of the enzyme were added to 10 ml of phosphate buffer, pH 7.8. After 10 minutes the reaction was stopped by adding 1.5 ml of H₂SO₄ (50%). The remaining hydrogen peroxide was filtered with 0.1M of KMnO₄ solution. Catalase activity was calculated on the basis that 1 ml of 0.1M of KMnO₄ solution corresponds to 1.7 ml of H₂O₂.

Superoxide Dismutase Activity

SOD activity was determined using the dianisidine method, which is based on the fact that under ultraviolet irradiation Riboflavin (Rb) is converted to an excited (ionised) state and therefore becomes able to attack reduced dianisidine (DH₂). The resulting flavin semiquinone further reduces the molecular oxygen and creates a superoxide radical. In the absence of SOD, the superoxide radical reduces the dianisidine radical in the reaction mixture. When SOD is available, the concentration of O₂ is very small; therefore dianisidine radicals interact with each other, producing one molecule of reduced (colourless) dianisidine and another molecule of oxidised (coloured) dianisidine. The higher the SOD activity, the more oxidised dianisidine is produced, with maximum absorption at 460 nm.

Incubation Medium:

| | | |
|---|---|---|
| 0.1 M phosphate buffer | 1 | ml |
| Riboflavin | 0.26 | ml |
| o-dianisidine | 0.04 | ml |
| Sample | 0.1 | ml |

The control sample contains a known amount of SOD. The samples were exposed to UV light for 10 minutes from a distance of 10 cm, cooled at room temperature and the optical density was measured.

The results, which are summarized in Table 22, showed that hepatectomy resulted in a distinct increase in DNA and RNA in the rat liver. The DNA content in the group treated with Compound 2 at a dose of 0.1 mM/kg or 0.2 MM/kg was 15% higher than that in animals undergoing hepatectomy alone, and the regenerating activity of the groups treated with Compound 3, Compound 5, Compound 6 or Compound 7 was higher than that of the group treated with 0.2 MM/kg Potassium orotate+Riboxin. The effectiveness of the compounds was in the following order: Compound 2 0.1 MM/kg>Compound 2 0.2 MM/kg>Compound 6 0.2 mM/kg>Compound 5 0.2 MM/kg>Compound 4 0.2 MM/kg>{Potassium orotate+Riboxin} 0.2 MM/kg.

The level of anti-oxidant protection was also assessed in terms of catalase and superoxidedismutase (SOD) activity. The importance of these enzymes is due to their physiological role related to oxidation processes involving molecular oxygen, hydrogen peroxide and oxygen radicals in metabolic changes. The changes in anti-oxidant activity are more pronounced with regard to catalase. A distinct stabilization of catalase activity was observed after treatment with Compound 2 at a dose of 0.2 MM/kg and Compound 6 at a dose of 0.2 MM/kg. These results were similar to those of the standard treatment group (Potassium orotate+Riboxin). An increase in repair processes was also observed with other compounds; however, their efficiency was slightly lower (Compound 2, 0.1 MM/kg>Compound 2, 0.2 MM/kg>Compound 5, 0.2 MM/kg>Compound 4, 0.2 M M/kg).

Therapeutic efficacy may be assessed in terms of the changes in the second anti-oxidant protection enzyme, SOD. Its activity in the liver of the experimental animals is reduced after hepatectomy. Administration of 0.2 MM/kg of Compound 2 or 0.2 MM/kg of Compound 6 resulted in a more pronounced stimulation of repair processes in the liver than when a conventional treatment (0.2 MM/kg Potassium orotate+Riboxin) was used. Positive changes in SOD activity were also found in the groups of animals which received Compound 2 at 0.1 MM/kg or 0.2 MM/kg and Compound 4 at 0.2 MM/kg after hepatectomy.

Therefore the efficacy of the compounds of the invention in terms of their ability to stabilize anti-oxidant enzyme activity is as follows: Compound 6 0.2 MM/kg>Compound 2 0.2 MM/kg>Compound 5 0.2 MM/kg (see Table 22).

TABLE 22
THE EFFECT OF 1,3-DIALKYL-4,5-BIS (N-METHYLCARBAMOYL) IMIDAZOLIUM SALTS
(COMPOUNDS III, V, VI AND VII) ON THE REGENERATION
OF LIVER AFTER A PARTIAL HEPATECTOMY

| | Groups of animals | | Hepatectomy + test components | | | | | |
|---|---|---|---|---|---|---|---|---|
| | | Hepatectomy | | | | | | Potassium |
| | Intact | alone | Compound 2 | Compound 2 | Compound 5 | Compound 6 | Compound 4 | orotate + |
| | animals | (control) | 0.1 mM/kg | 0.2 mM/kg | 0.2 mM/kg | 0.2 mM/kg | 0.2 mM/kg | Riboxin |
| Indications | n = 7 | n = 7 | n = 7 | n = 7 | n = 7 | n = 7 | n = 7 | 0.2 mM/kg |
| Level of | — | 100 | 150.5 | 134.9 | 120.3 | 105.0 | 128.3 | 144.7 |
| recovery* | | | | | | | | |
| RNA content | 5.42 ± 0.17 | 7.23 ± 0.25 | 7.07 ± 0.18 | 6.96 ± 0.39 | 7.46 ± 0.14 | 7.41 ± 0.16 | 7.14 ± 0.2 | 6.95 ± 0.35 |
| (mg/g of | | | | | | | | |
| tissue) | | | | | | | | |
| DNA content | 2.99 ± 0.21 | 3.65 ± 0.12 | 4.2 ± 0.37 | 4.1 ± 0.13 | 3.76 ± 0.29 | 3.88 ± 0.17 | 3.72 ± 0.09 | 3.48 ± 0.19 |
| (in mg/g of | | | | | | | | |
| tissue) | | | | | | | | |
| Catalase E | 34.0 ± 1.86 | 14.0 ± 0.9 | 18.0 ± 3.1 | 20.0 ± 1.92 | 17.3 ± 2.6 | 20.0 ± 2.7 | 18.0 ± 2.6 | 23.0 ± 3.1 |
| activity | | | | | | | | |
| μgM/min/g | | | | | | | | |
| tissue × 1000 | | | | | | | | |
| SOD | 90.8 ± 1.35 | 72.7 ± 5.9 | 78.9 ± 5.8 | 98.0 ± 4.6 | 103.0 ± 4.4 | 100.0 ± 4.7 | 90.0 ± 3.3 | 80.36 ± %.7 |
| activity | | | | | | | | |
| Units/g | | | | | | | | |
| tissue | | | | | | | | |
| Protein | 107.0 ± 4.9 | 93.2 ± 7.0 | 86.4 ± 9.8 | 94.8 ± 8.4 | 86.8 ± 6.9 | 93.0 ± 14.0 | 85.5 ± 10.6 | 91.5 ± 8.5 |
| content | | | | | | | | |
| Mg/g tissue | | | | | | | | |

*After partial hepatectomy some hepatomegaly is common.

### EXAMPLE 28

### Morphological study of the liver after partial hepatectomy and administration of Compound 2, Compound 4, Compound 5 and Compound 6

Morphological methods are highly informative in the assessment of repair processes. Histological tests of the liver after partial hepatectomy were performed using generally accepted methods. Microscopic sections were stained with hematoxylin and eosin using Van Gieson's methodology. Liver tests after partial hepatectomy were performed on 8 groups of animals, totalling 37 rats. All animals were examined one week after hepatectomy.

Histological examination indicated that the liver completely regenerated after partial hepatectomy in response to the test compounds. The following compounds were tested and showed no undesirable effects: Compound 2 0.1 MM/kg, Compound 6 0.2 MM/kg, Compound 4 0.2 MM/kg and, for comparison, a standard combination of Potassium orotate+Riboxin 0.2 MM/kg.

In contrast to the standard combination and in addition to hypertrophy of hepatocytes, test groups demonstrated an expansion of the growth areas with regenerating nuclei emerging in peripheral areas and proliferation of bile-duct epithelium. However, when Compound 2 0.2 MM/kg and Compound 5 MM/kg were used, some of the animals demonstrated hepatocyte hypertrophy combined with subacute hepatitis, with a pronounced sclerogenic change in the portal tracts and inflammatory infiltration correlating with dystrophic changes in hepatocytes.

The results indicate that Compound 2, which increases the organ regeneration completeness ratio by 34.9-50.5%, is the most effective of the compounds in activating repair processes in the liver, and exceeds the DNA content indicators in hepatectomised animals by 15%. The groups of animals treated with Compound 2, Compound 4, Compound 5 and Compound 6 had the highest regeneration activity compared to the combination of Potassium orotate+Riboxin. In terms of their effectiveness, these substances were as follows: Compound 2 0.1 MM/kg>Compound 2 0.2 MM/kg>Compound 6 0.2 MM/kg>Compound 5 0.2 MM/kg>Compound 5 0.2 MM/kg>{Potassium orotate+Riboxin} 0.2 MM/kg.

Our results in this model indicate that compounds of the invention are useful in stimulating liver regeneration following surgical resection.

### EXAMPLE 29

### Study of the clinical effect of Compound 2 in patients with chronic active viral hepatitis

45 patients aged between 18 and 32 years old with chronic active viral hepatitis were divided into 4 groups and kept under observation. All patients had received conventional therapy, usually potassium orotate, riboxin and interferon prior to commencement of the trial.

The first group (10 patients) received conventional therapy for 20 days. Assessment of clinical and biochemical indicators and serology data was carried out repeatedly for 20 days after the beginning of treatment.

The second group (10 patients) received both conventional treatment and prednisolone at a dose of 15 mg/kg for 20 days. In this group the clinical-biochemical and serological analysis took place before the treatment, and 10 days and 20 days after the beginning of treatment.

The third group (10 patients) received both conventional treatment and Compound 2 administered orally at a dose of 600 mg a day for 20 days. The control clinical-biochemical and serological analysis was carried out 20 days after the beginning of treatment.

The fourth group (15 patients) received prednisolone either orally or intravenously at a dose of 15 mg a day for 10 days; then prednisolone was stopped and replaced by Compound 2 at a dose of 600 mg a day for 10 days. In this group the first clinical-biochemical and serological analysis was carried out prior to the beginning of treatment, the second when treatment with prednisolone ceased and the final analysis after the full course of treatment with Compound 2.

Under such treatment with prednisolone a “rebound” phenomenon is observed, with an increase in immunoreactivity and a deceleration of replication of the virus. Prednisolone is conventionally prescribed at a dose of 20-30 mg a day over the course of 4 weeks, then it is abruptly stopped and after several days α-interferon is used. However, when prednisolone is employed over a long enough period of time (more than 2 weeks), replication of viruses is significantly activated, which makes the disease prognosis unfavourable.

Viral markers were found in all of the patients under observation. Chronic active hepatitis was diagnosed on the basis of primary clinical and laboratory data: the presence of astenovegetative syndrome, hepatomegaly with sclerosis of the liver, palpitations and tenderness, as well as spontaneous pain in the liver area, dyspeptic disturbances and icteric sclera. The following biochemical indicators were also considered: increased alanine transferase activity over the previous 6 months; increase in bilirubin or γ-globulin content in serum; as well as a decrease in sublimate titre and albumin content in the serum.

Our results with Compound 2 in patients with chronic active viral hepatitis have shown that this agent improves the clinical picture and normalizes biochemical indicators, particularly when combined with a preliminary course of prednisolone therapy; furthermore, it makes dyspepsia less pronounced and frequent, and consistently reduces alanine transferase activity.

### EXAMPLE 30

### Effect of Compound 2 on repair of bone tissue

A round or oval defect of the bone plate with a diameter of 2.5 mm was produced in the bone tissue of the rat lower jaw, using a dental drill under ether anaesthesia. In the post-operative period, the animals were kept in separate cages, with normal food and water ad libitum. The experiments were performed on 130 male rats weighing 180-200 g, with 10 rats in each group.

The animal test groups were treated with Compound 2 (50 or 100 mg/kg) or methyl uracil (50 mg/kg) 24 hours after the damage and on a daily basis for three months. The control group used animals with injured bone tissue, which did not receive any treatment. The following groups were used:

Group 1 intact animals;

Group 2 controls (bone plate defect; untreated) 30 days;

Group 3 bone plate defect+Compound 250 mg/kg 30 days;

Group 4 bone plate defect+Compound 2100 mg/kg 30 days;

Group 5 bone plate defect+Methyl uracil 50 mg/kg 30 days;

Group 6 controls (bone plate defect; untreated) 60 days;

Group 7 bone plate defect+Compound 250 mg/kg 60 days;

Group 8 bone plate defect+Compound 2100 mg/kg 60 days;

Group 9 bone plate defect+Methyl uracil 50 mg/kg 60 days;

Group 10 controls (bone plate defect; untreated) 90 days;

Group 11 bone plate defect+Compound 250 mg/kg 90 days;

Group 12 bone plate defect+Compound 2100 mg/kg 90 days;

Group 13 bone plate defect+Methyl uracil 50 mg/kg 90 days;

The repair pattern of the injured bone was assessed morphologically on tissue sections after 30, 60 and 90 days. The sections were examined by light microscopy at magnifications of ×15, ×100, ×200, and ×400.

Morphological analysis showed that bone tissue in the defect zones regenerated in response to Compound 2 or methyl uracil by way of primary healing with osteogenesis, following the pattern of appositional growth of mature compact bone tissue. There was not a single case of wound infection resulting in changes in the nature and timing of bone defect healing. The timing and nature of bone defect healing are consistent with literature data on bone tissue regeneration phases.

The best results in closing bone defects were shown by the group of animals who were given Compound 2 at 50 mg/kg over the course of three months, in which the defect was almost fully closed by the newly formed primary callus bone tissue, as shown in FIG. 7B, as compared with controls (FIG. 7A) and other test groups (FIGS. 7C and 7D).

### EXAMPLE 31

### Toxicity of 1,3-dialkyl-4,5-bis(N-methylcarbamoyl)imidazolium salts

The acute toxicity of 1,3-dialkyl-4,5-bis (N-methylcarbamoyl)imidazolium salts was assessed following intraperitoneal injection into 200 white male mice weighing 18 to 20 g. Dead animals were counted daily after the administration of the test substance. The total observation period was 14 days. LD₅₀ was calculated in accordance with Kerber's method (Belenky, 1963), and the results are shown in Table 23. Death was caused by respiratory failure; when a toxic dose of medication is administered, the animals lie motionless, then stop breathing and suffer cardiac arrest.

Compounds 1, 3, 4 and 7 all had very low toxicity, while even Compound 3 had an LD50 of over 500 mg/kg.

TABLE 23
Acute toxicity of 1,3-dialkyl-4,5-bis(N-methylcarbamoyl)-
imidazolium salts (LD₅₀)

| | LD₅₀ |
|---|---|
| Medication | mg/kg |
| Compound (1) | 1833 |
| Compound (2) | 764 |
| Compound (3) | 1292 |
| Compound (4) | 1221 |
| Compound (7) | 1221 |

We found that compound 2 is effective at doses of 10 to 20 mg/kg for prolonged oral and/or parenteral administration. In these studies, the LD₅₀ for rats was 1210 mg/kg, and for mice, the LD₅₀ was 1920 mg/kg when given intraperitoneally. For oral administration the LD₅₀ for rats and guinea pigs was more than 20000 mg/kg. It is contemplated that in clinical use, treatment using oral tablets will take two to three weeks, with each patient receiving three tablets containing 0.6 g active compound per day.

Chronic toxicity studies in animals over a period of six months indicated that compound 2 had no irritating or allergic action, does not act on the central nervous system, and has no teratogenic, mutagenic or carcinogenic immunosuppressive activity. No effect on body weight, weight of internal organs, structure or function of internal organs or blood indicators was observed. These studies were performed in mice (40 mg/kg per day, given subcutaneously), in rats (40 mg/kg per day, given subcutaneously) and in dogs (10 mg/kg or 40 mg/kg per day, given orally).

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Belenky M. L. Elements of quantitative assessment of pharmaceutical effect (1963)
Gaivoronskaya V. V., Okovity S. V., Shustov E. B., Smirnov A. B., Experimental and clinical Pharmacology, 2000, T 63, No. 5, P. 34-36
Higgins G. M. and Anderson R. M., Arch. Pathol, 1931, V.12. N.1, P. 188-202
Katzung G. B., Dermatological pharmacology /Basic and clinical pharmacology: 1998 Volume 2 pp. 552-571
Kuzin M. I., Kostyuchenko B. M., Karlov V. A. Summary review of wound related theories. //Wounds and wound infection.—M., Medicine, 1981, pp. 13-54
Liubimov, B. I. et al 1999. Methodological instructions with regard to the assessemnt of the allergic characteristics of pharmacuetical substances in “Manual on Experimental Pre-chemical study of new pharmaceutical substances p 234-241
Mashkovsky M. D., Medicinals stimulating metabolic processes// Medicinals. 1998, vol. 2, pp. 168-192
Piotrovsky L. B., Marasanova N. Y., Khromov-Borisov N. V., Moreva E. V., Sapronov N. S., Novikov V. P., Kovaleva V. A. Russian Patent No. 1075668: Benzenesulphonates of 1,3-dialkyl-4,5-bis (N-methylcarbamoyl)-imidazolium, stimulating tissue energy exchange
Rychnev V. E., Frolov V. M., Regeneration stimulators used in treatment of viral hepatitis and other liver diseases, Voronezh State University Press, Voronezh, 1984
Sarkisov D. S., Paltsyn A. A., Musikant L. I.: Morphology of the wound healing process, Wounds and wound infection. Moscow.: 1981, pp. 688-695
Scheuplein R. J.: Mechanism of percutaneous absorption. J. Investig. Dermatology, 1965, v.45, N5, P. 334-345
Solopaev B. P., Regeneration of normal and pathological alterations of the liver, Volgo-Viat. Book Publishers, Gorky 1980
Swingle, K. F., Shideman, F. E., 1972 J. Pharmacol. Exp. Therap. 183 226-234
Sriprachya-Anunt S., Fitzpatrick R. E., Goldman M. P., Smith S. R. Infections complicating pulsed carbon dioxide laser resurfacing for photoaged facial skin.//Dermatologic Surgery, 1997, 23(7): 527-35
Winter, C. A., Risley F. A. Nuss, G. N., 1962 Proc. Natl. Exp. Biol. Med. 3 544-547
Zapadnyuk I. P., Zapadnyuk V. I., Zakharia E. A., Zapadnyuk B. V.//Laboratory animals. -Kiev. Vyscha shkola, 1983, pp. 248-250, 254-255

1. A method of promoting tissue repair or wound healing, comprising the step of administering an effective amount of a 1,3-dialkyl-4,5-bis (optionally N-substituted carbamoyl)imidazolium salt to a subject in need of such treatment.

2. A method according to claim 1, wherein said method reduces inflammation in said subject.

3. A method according to claim 1, in which the 1,3-dialkyl-4,5-bis (optionally N-substituted carbamoyl)imidazolium salt is a compound of formula I 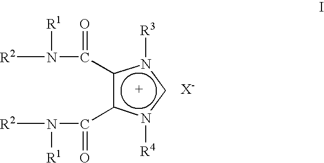
in which R¹ and R² are the same or different, and each is selected from the group consisting of hydrogen and a linear or branched alkyl group of 1 to 6 carbon atoms, which may optionally be substituted by an amino, substituted or unsubstituted aminomethyl, nitro, hydroxyl, halogen, carboxy, or carboxylic acid amide group;
R³ and R⁴ are the same or different, and each is a substituted or unsubstituted linear or branched alkyl group of 1 to 6 carbon atoms; and
X⁻ is a pharmaceutically acceptable inorganic or organic anion selected from the group consisting of chloride, bromide, iodide, sulphate, nitrate, phosphate, perchlorate, formate, acetate, fumarate, malate, malonate, citrate, benzoate, salicylate, benzenesulphonate, methylsulphonate, p-toluenesulphonate, gentisate, and naphthalene-8-sulphonate.

4. A method according to claim 3, in which at least one of R³ and R⁴ is unsubstituted.

5. A method according to claim 4, in which both R³ and R⁴ are unsubstituted.

6. A method according to claim 4, in which where R¹ or R² is substituted with a substituted sulphonamide, the substituent is an alkyl chain of 1 to 6 carbon atoms.

7. A method according to claim 3, in which R¹ and R² are different; and R³ and R⁴ are the same or different, and is each independently an alkyl group with 1 to 6 carbon atoms.

8. A method according to claim 7, in which R³ and R⁴ are both alkyl groups of 1 to 4 carbon atoms.

9. A method according to claim 8, in which R³ and R⁴ are both methyl or both ethyl, or one of R³ and R⁴ is methyl and the other is ethyl.

10. A method according to claim 8, in which R³ is methyl and R⁴ is ethyl.

11. A method according to claim 3, in which X⁻ is benzenesulfonate, benzoate, salicylate, or gentisate.

12. A method according to claim 11, in which X⁻ is benzenesulphonate.

13. A method according to claim 3, in which X⁻ is an inorganic anion selected from the group consisting of chloride, bromide, and iodide.

14. A method according to claim 3, in which the subject is suffering from epithelial damage to skin or mucous tissue, caused by erosions, ulcers, chronic injury, infection, trauma or surgery.

15. A method according to claim 1, in which the subject is suffering from a condition selected from the group consisting of traumatic wounds, surgical wounds, burns, dehisced surgical incisions, grafts, diabetic ulcers, varicose ulcers, decubitus ulcers (bedsores), trophic ulcers, tropical ulcers, steroid ulcers, indolent ulcers, oral or pharyngeal ulcers, aphthous ulcers, and corneal ulcers; and cervical erosions.

16. A method according to claim 1, in which the subject is suffering from a condition selected from the group consisting of gastric or duodenal ulcers, and ulcerative colitis.

17. A method according to claim 1, in which the subject is suffering from a condition selected from the group consisting of myocardial damage, liver damage and bone damage.

18. A method according to claim 17, wherein said method stimulates liver regeneration in said subject.

19. A method according to claim 15, wherein said method reduces or prevents scar formation in said subject.

20. A method according to claim 16, wherein said method treats ulcerative colitis in said subject.

21. A method according to claim 15, wherein said method treats oral or pharyngeal ulceration in said subject.

22. A method according to claim 17, wherein said method treats hepatic cirrhosis or chronic active hepatitis in said subject.

23. A method according to claim 16, wherein said method treats gastric or duodenal ulcers in said subject.

24. A method according to claim 17, wherein said method treats myocardial infarction in said subject.

25. A method according to claim 17, wherein said method stimulates bone repair in said subject.

26. A method according to claim 1, in which the 1,3-dialkyl-4,5-bis (optionally N-substituted carbamoyl) imidazolium salt is selected from the group consisting of
1,3-dimethyl-4,5-bis(N-methylcarbamoyl)imidazolium benzenesulfonate,
1-methyl-3-ethyl-4,5-bis(N-methylcarbamoyl)imidazolium benzenesulfonate,
1,3-diethyl-4,5-bis(N-methylcarbamoyl)imidazolium benzenesulfonate,
1-methyl-3-ethyl-4,5-bis(N-methylcarbamoyl)imidazolium benzoate,
1-methyl-3-ethyl-4,5-bis(N-methylcarbamoyl)imidazolium salicylate,
1-methyl-3-ethyl-4,5-bis(N-methylcarbamoyl)imidazolium gentisate, and
1-methyl-3-ethyl-4,5-bis(N-methylcarbamoyl)imidazolium chloride.

27. A compound of formula I 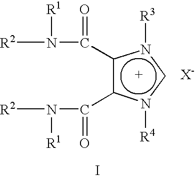
in which R¹ and R² are the same or different, and each is selected from the group consisting of hydrogen and a linear or branched alkyl group of 1 to 6 carbon atoms, which may optionally be substituted by an amino, substituted or unsubstituted aminomethyl, nitro, hydroxyl, hydrogen, carboxy, or carboxylic acid amide group;
R³ and R⁴ are the same or different, and each is a substituted or unsubstituted linear or branched alkyl group of 1 to 6 carbon atoms; and
X⁻ is a pharmaceutically acceptable inorganic or organic anion selected from the group consisting of chloride, bromide, iodide, sulphate, nitrate, phosphate, perchlorate, formate, acetate, fumarate, malate, malonate, citrate, benzoate, salicylate, benzenesulphonate, methylsulphonate, p-toluenesulphonate, gentisate, and naphthalene-8-sulphonate,
with the proviso that when X⁻ is benzenesulphonate, R¹ is hydrogen and R² is methyl, R³ and R⁴ are not methyl or ethyl.

28. A compound according to claim 27, in which at least one of R³ and R⁴ is unsubstituted.

29. A compound according to claim 28, in which both R³ and R⁴ are unsubstituted.

30. A compound according to claim 27, in which where R¹ or R² is substituted with a substituted sulphonamide, the substituent is an alkyl chain of 1 to 6 carbon atoms.

31. A compound according to claim 27, in which R¹ and R² are different; and R³ and R⁴ are the same or different, and are each independently an alkyl group with 1 to 6 carbon atoms.

32. A compound according to claim 31, in which R³ and R⁴ are alkyl groups of 1 to 4 carbon atoms.

33. A compound according to claim 32, in which R³ and R⁴ are both methyl or both ethyl, or one of R³ and R⁴ is methyl and the other is ethyl.

34. A compound according to claim 33, in which R³ is methyl and R⁴ is ethyl.

35. A compound according to claim 27, in which X⁻ is benzenesulfonate, benzoate, salicylate, or gentisate, with the proviso that when X⁻ is benzenesulphonate, R¹ is hydrogen and R¹ is methyl, R³ and R⁴ are not methyl or ethyl.

36. A compound according to claim 35, in which X⁻ is benzenesulphonate.

37. A compound according to claim 27, in which X⁻ is an inorganic anion selected from the group consisting of chloride, bromide, and iodide.

38. A compound according to claim 27, selected from the group consisting of
1-methyl-3-ethyl-4,5-bis(N-methylcarbamoyl)imidazolium benzoate,
1-methyl-3-ethyl-4,5-bis(N-methylcarbamoyl)imidazolium salicylate,
1-methyl-3-ethyl-4,5-bis(N-methylcarbamoyl)imidazolium gentisate, and
1-methyl-3-ethyl-4,5-bis(N-methylcarbamoyl)imidazolium chloride.

39. A compound according to claim 27, together with a pharmaceutically or veterinarily acceptable carrier.

40. A compound according to claim 39, in which the carrier is adapted for topical administration.

41. A compound according to claim 39, in which the carrier is adapted for oral, buccal or sub-lingual administration.

42. A method of synthesis of a compound according to claim 27, comprising the step of subjecting a 1-alkylimidazole-4,5-bis(optionally N-substituted carbamoyl)imidazole to alkylation (quaternization) with an alkyl benzenesulfonate to produce the corresponding imidazolium benzenesulfonate, and optionally replacing the benzenesulfonate anion by ion exchange, in which the imidazole moiety is as defined in claim 27.

43. A method of promoting tissue repair, promoting wound healing or reducing inflammation in a subject, comprising administering to said subject a 1,3-dialkyl-4,5-bis (optionally N-substituted carbamoyl)imidazolium salt in an amount effective to promote tissue repair, promote wound healing or reduce inflammation in said subject.

44. A composition comprising a compound according to claim 27 and a pharmaceutically or veterinarily acceptable carrier.

45-50. (canceled)
